(19) United States
(12) Patent Application Publication
(10) Pub. No.: US 2003/0148954 A1
(43) Pub. Date: Aug. 7, 2003

(54) Agents and methods for modulating activator protein-1-mediated cellular processes

(75) Inventors: Emery H. Bresnick, Middleton, WI (US); Jason E. Norton, Madison, WI (US); Jianlin Chu, Madison, WI (US)

Correspondence Address: GODFREY & KAHN, S.C., 780 N. WATER STREET, MILWAUKEE, WI, 53202, US

(73) Assignee: Wisconsin Alumni Research Foundation

(21) Appl. No.: 10/287,196

(22) Filed: Nov. 4, 2002

Related U.S. Application Data
(60) Provisional application No. 60/335,379, filed on Nov. 2, 2001.

(51) Int. Cl. A61K 38/17; C12Q 1/68; G01N 33/53; G01N 33/567

(52) U.S. Cl. 514/12; 435/6; 435/7.2

(57) ABSTRACT

Agents for modulating AP-1 mediated gene expression are provided. The modulating agents comprise an internalization moiety and: (1) a peptide sequence isolated from the intracellular domain of Notch-1 (NIC-1); or (2) a peptide analogue or peptidomimetic of the NIC-1 peptide sequence. Methods of using the modulating agents for modulating AP-1 mediated gene expression in a variety of contexts (e.g., for modulating inflammatory and immunosuppressive activities) are provided.

### CROSS-REFERENCE TO RELATED APPLICATIONS

This application claims benefit under 35 U.S.C. §119 to U.S. provisional application No. 60/335,379, filed on Nov. 2, 2001, which is specifically incorporated herein by reference in its entirety for all purposes.

### STATEMENT AS TO RIGHTS TO INVENTIONS MADE UNDER FEDERALLY SPONSORED RESEARCH AND DEVELOPMENT

This work was supported in part by a grant from the National Institutes of Health Grant DK50107. The Government of the United States of America may have certain rights in this invention.

### FIELD OF THE INVENTION

The present invention relates generally to agents and methods for use in modulating Activator Protein-1 (AP-1) mediated cellular processes. The invention is more specifically related to: (1) modulating agents capable of affecting AP-1-mediated transcriptional; (2) methods of identifying such modulating agents; and (3) methods employing such agents.

### BACKGROUND OF THE INVENTION

The highly conserved Notch signaling pathway controls cell-fate decisions in organisms as diverse as insects, nematodes, and mammals (1-3). Examples of biological processes regulated by Notch signaling include neurogenesis (4,5), hematopoiesis (6), vasculogenesis (7), and cortical neurite growth (5). Four paralogs of Notch, Notch 1-4, and five Notch ligand genes, Jagged-1, Jagged-2, Delta-1, Delta-like 1, and Delta-like 3, have been identified in vertebrates (8).

Two modes of Notch signaling have been proposed, involving either interaction of the intracellular domain of Notch (NIC) with CSL (CBF1/RBP-Jκ, Su(H), and Lag-1) repressor proteins (9-11) or a CSL-independent mechanism (12,13). In the canonical CSL-dependent mechanism, activation of a Notch transmembrane receptor by a transmembrane ligand on a neighboring cell results in two consecutive proteolytic cleavages, allowing for release and nuclear translocation of NIC (14-16). Nuclear NIC physically interacts with CSL bound with sequence-specificity to promoters of target genes (11). Additional components such as mastermind (17,18), and SKIP (19) assemble into the NIC/CSL nucleoprotein complex and are important for transactivation. CSL-independent signaling apparently also involves transcriptional regulation (12), but there is still much to be learned about the requisite components and the underlying mechanisms.

Since Notch has multiple conserved domains with the potential to be protein docking sites, Notch might act as a scaffold to assemble complexes containing components of the Notch and other signaling pathways. As with any complex signaling system, physiological functions mediated by Notch are likely to depend on how Notch signals integrate with signals emanating from other pathways. Indeed, Notch signaling interacts with multiple signaling pathways including Ras (13,20-23), Wnt (24-26), T-cell receptor (27), granulocyte colony stimulating factor (28), granulocyte-macrophage colony stimulating factor (28), and NF-κB (29-32).

Multiple lines of evidence support the existence of physiological crosstalk between the Notch and Ras pathways. Notch mutants in Drosophila have elevated levels of the Ras-regulated stress-activated kinase JNK (13), suggesting negative crosstalk between Notch and JNK pathways. In addition, Notch-1 and Notch-2 inhibit the E47 transcription factor, and this involves inhibition of Ras signaling, which is required for E47 activity (21). Moreover, during vulval development in C. elegans, Notch-mediated transcriptional activation of the MAPK phosphatase LIP-1, which counteracts Ras-dependent MAPK signaling, establishes the basis for opposing Notch and Ras signals (23).

In contrast, Ras signals are required for anchorage-independent growth of cancer cell lines derived from Notch-4-expressing transgenic mice (22). Although the consequences of interactions between Notch and Ras are just beginning to be investigated, such interactions would likely affect the activity of the transcription factor AP-1, a major nuclear target of Ras.

AP-1 consists of homodimers of Jun family members or heterodimers of Jun and Fos proteins (33). Growth factors, cytokines, and tumor promoters activate AP-1 as an integral step in their mechanism of action (34), establishing a crucial role for AP-1 in many cellular processes including proliferation, differentiation, and survival. Dysregulation of AP-1 is a prototypical mechanism of tumor promotion (35). Disruption of Notch signaling can also transform cells (36,37) and has been hypothesized to cause leukemogenesis (reviewed in 38,39).

The mechanism of Ras-dependent AP-1 activation involves phosphorylation of c-Jun and Jun family members on amino-terminal serines (serines 63 and 73 for c-Jun) (40). These modifications are often mediated by JNK (41), but p38 can also catalyze phosphorylation at these sites (42). Phosphorylation of threonine 231 and serine 249 near the DNA binding domain of c-Jun represses DNA binding, and dephosphorylation confers high-affinity binding (43). c-Jun phosphorylated at serines 63 and 73 interacts with the coactivator CBP/p300 (44). CBP/p300 confers transcriptional activation via histone and nonhistone protein acetylation (45,46), although the mechanism for how AP-1 utilizes CBP/p300 is unclear. An additional AP-1 coactivator is Jab1 (47), a component of the COP9 signalsome complex (48), which stabilizes DNA-bound AP-1 complexes (47). The AP-1 stimulatory activity of Jab1 has been reported to be JNK-dependent (49) and -independent (50) in different systems. Thus, AP-1 is a dynamically regulated nuclear effector of Ras and integrates diverse cellular signals.

Recently, a correlation between Notch-1 activity and AP-1 activity in carcinoma cells was reported. See Talora et al., (2002) Genes & Development 16: 2252-2263. It was shown that the expression of endogenous Notch-1 is markedly reduced in a panel of cervical carcinoma cells and Notch-1 expression is reduced or absent in invasive cervical cancers. Conversely, expression of activated Notch-1 causes strong growth inhibition of human papillomaviruses (HPV)-positive, but not HPV-negative, cervical carcinoma cells, but exerts no such effects on other epithelial tumor cells. It was further observed that increased Notch-1 signaling, but not Notch-2, causes a dramatic down-modulation of HPV-driven transcription of the E6/E7 viral genes, through suppression of AP-1 activity. Notch-1 was therefor observed to exert specific protection against HPV-induced transformation in an AP-1 dependent manner.

The recent observations by Talora et al. further substantiate a prior discovery by the present inventors, to be described below, directed to previously unreported crosstalk between Notch-1 and AP-1. See Chu et al., (2002) J. Biol. Chem. 277: 7587-7597. In this regard, the modulation of AP-1 transactivation by Notch-1 serves as the basis for the invention to be described herein. In light of the above-described findings and the present inventors pioneering discovery, and because of previous additional studies demonstrating AP-1's integral role in cellular processes including normal and dysfunctional proliferation and differentiation, AP-1 modulating therapeutics based on the crosstalk between Notch-1 and AP-1 represent an extremely desirable new area of study.

### SUMMARY OF THE INVENTION

The present invention is based on the unique observation that the intracellular domain of human Notch-1 (NIC-1) strongly represses AP-1 mediated transactivation. Given the growing array of biological processes that Notch-1 and AP-1 control, crosstalk between Notch-1 and AP-1 has important physiological and pathophysiological implications and, specifically, is an avenue for the development of modulating agents capable of modulating AP-1 transactivation in a selective manner. Accordingly, the present invention provides agents, methods of identifying agents, and methods for modulating AP-1 mediated transactivation utilizing the agents.

In one embodiment, the present invention is a modulating agent for modulating AP-1 mediated cellular processes in a cell including an internalization moiety and one or more of: (a) an isolated polypeptide from the intracellular domain of Notch-1 (NIC-1) which is capable of modulating an AP-1 mediated cellular process; or (b) a peptide analog or peptidomimetic of the isolated polypeptide of (a) which is capable of modulating an AP-1 mediated response.

A modulating agent of one embodiment includes an isolated polypeptide comprising the RAM domain of NIC-1 (SEQ ID NO:1). The RAM domain is a 90 amino acid region within NIC-1 which was previously identified as necessary for CSL binding and CSL-dependent activation. The RAM domain spans amino acids 1759-1848 of the human Notch-1 polypeptide. The ability of the RAM domain to modulate AP-1 mediated transactivation constitutes a previously undescribed activity of the highly conserved RAM domain. In other embodiments, modulating agents include partial amino acid sequences of the RAM domain which are capable of modulating AP-1 transactivation. Examples of such modulating agents include those including or derived from RAM domain amino acid sequences 1759-1819 (SEQ ID NO:2), 1820-1848 (SEQ ID NO:3), and 1759-1841 (SEQ ID NO:4).

In certain embodiments of the invention, the internalization moiety present in a modulating agent is a peptide localization signal for directing a modulating agent to the cytoplasm of a cell and/or the nuclear compartment. In other embodiments, a liposome may serve as the internalization moiety.

The invention also encompasses methods for modulating AP-1 mediated cellular processes in a cell, comprising contacting a cell with a modulating agent that comprises one or more of: (a) an isolated polypeptide from the intracellular domain of Notch-1 (NIC-1) which is capable of modulating an AP-1 mediated cellular process; or (b) a peptide analog or peptidomimetic of the isolated polypeptide of (a) which is capable of modulating an AP-1 mediated response. AP-1 mediated cellular processes are thereby modulated within a cell. In one embodiment, a method of modulating AP-1 transactivation is effectuated by a recombinant nucleic acid present within the cell which encodes a modulating agent.

The invention is also a method of treating a disease state in a patient including the step of administering to the patient a therapeutically-effective amount of a modulating agent as described herein. The disease state may be, in particular, an inflammatory response, an immune response, or a cancerous growth such as a leukemia. The method of treatment may include the step of administering pharmaceutical compositions comprising pharmaceutically acceptable carriers and a modulating agent.

The present invention is also directed to methods of identifying Notch-1 intracellular (NIC-1) domain-derived modulating agents effective in modulating AP-1 mediated transcription. Such methods include the steps of: (a) obtaining a cell line or organism transformed with a reporter gene operably linked to an AP-1 responsive element; (b) contacting said transformed cell line or organism with: (i) a polypeptide including a portion of the intracellular domain of Notch-1 (NIC-1); or (ii) a peptide analog or peptidomimetic of the polypeptide of (i); and (c) assaying the activity of the reporter gene wherein a statistically significant difference in the activity between the reporter gene in a transformed cell line or organism contacted with said portion of the NIC-1 domain, analog, or peptidomimetic thereof and the reporter gene in a transformed cell line or organism not contacted with said portion of the NIC-1 domain, analog, or peptidomimetic thereof correlates with the identification of a modulating agent.

Other objects, features and advantages of the present invention will become apparent after review of the specification, claims and drawings.

### BRIEF DESCRIPTION OF THE DRAWINGS

FIG. 1. NIC-1 specifically represses AP-1-mediated transactivation in K562 cells. K562 cells were transiently cotransfected with pBabe or pNIC-1 and reporter vectors containing five Gal4 (p5×Gal4luc) or four CBF1 (p4×CBF1luc) binding sites (A), collagenase promoter (p1×AP1luc) (B), two AP-1 binding sites upstream of a human β-globin promoter [pβ106(AP 1)2luc] (C), three NF-κB binding sites upstream of a minimal promoter (p3×κBluc) (D), a luciferase reporter driven by a human Aγ-globin promoter (pGL3γluc) (E), or a constitutively active β-galactosidase reporter containing the CMV enhancer (pCMVβgal) (F). AP-1-dependent reporter activity was induced by TPA treatment (5 nM, 12 h). The luciferase and β-galactosidase activities were normalized by the protein content of the lysate. Each graph depicts averaged data from at least three independent transient transfection experiments (mean+/−S.E.).

FIG. 2. A component of TPA-inducible AP-1 activity is resistant to NIC-1. K562 cells were transiently cotransfected with pBabe or pNIC-1 and the reporter vector containing one AP-1 (p1×AP1luc) binding site. AP-1 reporter activity was induced by treatment of cells with the indicated concentrations of TPA for 16 h. The luciferase activity was normalized by the protein content of the lysate (mean+/−S.E., n=3).

FIG. 3. NIC-1 completely inhibits H-Ras(12V)-inducible AP-1 activity. K562 cells were transiently cotransfected with pBabe, pNIC-1, or pBabe-H-Ras(12V) and either the p1×AP1luc or p4×CBF1luc reporter vectors. Luciferase activity was normalized by the protein content of the lysate. The graph depicts averaged data from three independent transient transfection experiments (mean+/−S.E.).

FIG. 4. NIC-1 represses AP-1-mediated transactivation in HeLa cells. HeLa cells were transiently cotransfected with pbabe or pNIC-1 and reporter vectors containing four CBF1 (p4×CBF1luc) or one AP-1 (p1×AP1luc) binding sites. AP-1 reporter activity was induced by TPA treatment (5 nM, 16 h). The luciferase activity was normalized by the protein content of the lysate. The graph depicts averaged data from three independent transient transfection experiments (mean+/−S.E.).

FIG. 5. Endogenous AP-1 target genes are deregulated by NIC-1. A, RNA from pools of K562-Babe and K562-NIC-1 cells treated with vehicle (DMSO) or 5 nM TPA for 12 h was analyzed by Northern blotting with IL-8, MMP1, IκBα, and GAPDH probes. B, quantitative analysis. Relative expression values were determined by analysis of Northern blots with a PhosphorImager. The levels of IL-8, MMP1, and IκBα transcripts were normalized by the level of GAPDH transcripts to yield the relative expression values. The quantitative data represent analysis of RNA from three to seven pools of K562-Babe and K562-NIC-1 cells, respectively (mean+/−S.E.).

FIG. 6. Similar concentration requirement for NIC-1-mediated activation of CSL-dependent transactivation and AP-1 repression. K562 cells were transiently transfected with either the AP-1-responsive reporter (p1×AP1luc) or CBF 1-responsive reporter (p4×CBF1luc) in the presence of increasing amounts of NIC-1 expression vector. AP-1 reporter activity was induced by TPA treatment (5 nM, 12 h). The luciferase activity was normalized by the protein content of the lysate. Normalized luciferase activity expressed as the percentage of the maximal response was plotted against NIC-1 concentration. The graph depicts averaged data from five independent transient transfection experiments (mean+/−S.E.).

FIG. 7. Overlapping amino acid sequence determinants within the RAM domain of NIC-1 confer CSL-dependent activation and AP-1 repression. A, schematic representation of Myc-tagged wild-type NIC-1 and NIC-1 mutants. B, detection of wild type NIC-1 and NIC-1 mutants by Western blotting. A blank vector or NIC-1 expression vectors were introduced into K562 cells by retroviral infection. Cell lysates were immunoprecipitated with anti-NIC-1 antibody, and bands were detected by Western blotting with anti-Myc antibody. The bracket denotes bands representing wild-type NIC-1 and mutants. C, The blot was reprobed with anti-CBF1 antibody. D, transient transfection analysis. K562 cells were transiently transfected with either CBF1 or AP-1 reporter vectors and pBabe, wild type NIC-1 or NIC-1 mutants. AP-1 reporter activity was induced by TPA treatment (5 nM, 16 h). Luciferase activity was normalized by the protein content of the lysate. The graph depicts averaged data from four independent transient transfection experiments (mean+/−S.E.). E, Sequence conservation of the RAM domain. Note that the human and mouse RAM domain sequences differ by only a single amino acid.

FIG. 8. Predominant nuclear localization of NIC-1 correlates with AP-1 repression. A, schematic representation of NIC-1 constructs containing NLS (NIC-1/NLS) or NES (NIC-1/NES). B, NIC-11NES is partially excluded from the nucleus. Transiently transfected HeLa cells were processed for IF as described in Experimental Procedures. The transfected wild-type and modified NIC-1 constructs are indicated at the top of each column. NIC-1 proteins were visualized with Cy3 (top row); nuclei were stained with DAPI (middle row); merged Cy3 and DAPI images are shown in the bottom row (Cy3+DAPI). C, transient transfection analysis. K562 cells were transiently transfected with either CBF1 or AP-1 reporters and wild type NIC-1 or NIC-1 derivatives. AP-1 reporter activity was induced by TPA treatment (5 nM, 16 h). The luciferase activity was normalized by the protein content of the lysate. The graph depicts averaged data from three independent transient transfection experiments (mean+/−S.E).

FIG. 9. NIC-1 does not affect ERK1/2, p38/MAPK, JNK, and c-Jun phosphorylation events associated with the active signaling state. A, pools of K562-Babe and K562-NIC-1 cells were pretreated with 5 nM TPA or the solvent (DMSO) for 30 min. Cells (1×10⁶) were lysed by boiling in SDS sample buffer and 10% of total protein was analyzed by Western blotting by using phospho-specific antibodies as indicated. After incubation with secondary antibodies, antigen/antibody complexes were visualized by chemiluminescence. Blots were stripped and reprobed with antibodies reacting with total proteins as indicated. B, stably transfected K562 cells (1×10⁶) were stimulated with 5 nM TPA for 0, 5, 10, or 20 min. At the indicated times, cell lysates were prepared, and proteins were resolved by SDS-polyacrylamide gel electrophoresis (10%), and subjected to immunoblotting with c-Jun (Ser73) antibodies. The blots were stripped and reprobed with anti-c-Jun antibody. The blots in A and B are representative of results from analysis of four pools of K562-Babe and K562-NIC-1 cells, respectively.

FIG. 10. NIC-1 does not affect AP-1 DNA binding activity in vitro. Pools of K562-Babe and K562-NIC-1 cells were treated with 5 nM TPA or vehicle for 2 hours. AP-1 DNA binding activity in nuclear extracts (5 μg) was measured by EMSA using a double-stranded oligonucleotide containing a single binding site for AP-1. Lane 1, probe incubated with no nuclear extract. The specific incubation conditions for other lanes are indicated at the bottom of the figure. Note that preincubation of the extract with c-Jun and c-Fos antibodies reduced the levels of complex formation, whereas an equivalent amount of IgG had no effect. In addition, the amount of complex formed was reduced by a stoichiometric excess of unlabeled AP-1 oligonucleotide but not USF oligonucleotide.

### DETAILED DESCRIPTION OF THE INVENTION

Before the present agents and methods are described, it is understood that this invention is not limited to the particular agents, compositions, methodology, protocols, cell lines, vectors, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a cell” includes a plurality of such cells, reference to the “vector” is a reference to one or more vectors and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the compounds, polypeptides, polynucleotides, cell lines, vectors, and methodologies which are reported in the publications which might be used in connection with the invention. Nothing herein is to be construed as an admission that the invention is not entitled to antedate such disclosure by virtue of prior invention.

The practice of the present invention will employ, unless otherwise indicated, conventional techniques of chemical synthesis, rational drug design, cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Abbreviations used throughout include: AP-1, activator protein-1; CBF1, C promoter binding factor 1; CMV, cytomegalovirus; CSL, CBF1/RBP-Jκ, Su(H), and Lag-1; EMSA, electrophoretic mobility shift assay; ERK, extracellular regulated kinase; IF, indirect immunofluorescence; IL-8, interleukin-8; JNK, c-Jun amino terminal kinase; luc, luciferase; MMP 1, matrix metalloproteinase 1; NIC-Notch intracellular domain; NES, nuclear export signal; NLS, nuclear localization signal; PBS, phosphate buffered saline; RAM, RBP-Jκ-associated molecule; RLU, relative light units; and SDS-PAGE, SDS polyacrylamide gel electrophoresis.

The Invention

The present invention provides modulating agents and methods for modulating AP-1 mediated cellular processes. The present invention is based, in part, upon the discovery that the intracellular domain of Notch-1 (NIC-1) contains amino acid sequences (in particular, the 90 amino acid RAM domain (SEQ ID NO:1) and sequences therein) which are capable of modulating AP-1 mediated cellular processes, in particular, transcriptional activation.

The term “modulating agent,” as used herein, refers to a composition comprising an internalization moiety and one or more of: (1) an isolated polypeptide from the intracellular domain of Notch-1 (NIC-1) which is capable of modulating an AP-1 mediated cellular process; or a peptide analog or peptidomimetic of the polypeptide of (1) which is capable of modulating an AP-1 mediated cellular process. In general, the internalization moiety present in the modulating agent may be associated with the polypeptide, analog, or peptidomimetic in a covalent or noncovalent fashion.

In one embodiment, the polypeptide is the amino acid sequence representing the RAM domain of human Notch-1. The RAM domain spans amino acid sequences 1759-1848 of the human Notch-1 polypeptides. The RAM domain, as further defined and described in the Example section below, has been discovered by the present inventors to exhibit a previously unreported ability to modulate AP-1 mediated transactivation. Specifically, the RAM domain, and portions thereof, are capable of inhibiting AP-1 transactivation such that AP-1 dependent cellular processes are diminished or prevented. Partial amino acid sequences of the RAM domain have utility in modulating AP-1 transactivation and certain embodiments may be based on these partial sequences. Elucidation of these partial sequences is described in the Example section below and includes the RAM domain amino acid sequences 1759-1819 (SEQ ID NO:2), 1820-1848 (SEQ ID NO:3), and 1759-1841)SEQ ID NO:4). Peptide-based modulating agents described herein may, but need not, contain additional amino acid residues from those capable of modulating AP-1 mediated transactivation. Such additional residues may flank the modulating sequences in a native Notch-1 molecule (i.e., may be adjacent to that sequence in a native Notch-1 molecule) or may represent sequences not naturally-flanking the sequences responsible for AP-1 modulation. Flanking residues may be present on the N-terminal and/or C-terminal side of a peptide-based modulating agent sequence and may, although not necessarily, act to facilitate internalization, cyclization, purification or other manipulation of the peptide-based modulating agent. Modulating agents may further be associated (covalently or noncovalently) with a targeting agent, drug, solid support and/or detectable marker.

Certain preferred modulating agents comprise a peptide in which at least one terminal amino acid residue is modified (e.g., the N-terminal amino group is modified by, for example, acetylation or alkoxybenzylation and/or an amide or ester is formed at the C-terminus). The addition of at least one such group to a linear or cyclic peptide modulating agent may improve the activity of the agent, enhance cellular uptake and/or impair degradation of the agent

Modulating agents, or peptide portions thereof, may be linear or cyclic peptides. A “linear” peptide is a peptide or salt thereof that does not contain an intramolecular covalent bond between two non-adjacent residues. The term “cyclic peptide,” as used herein, refers to a peptide or salt thereof that comprises an intramolecular covalent bond between two non-adjacent residues, forming a cyclic peptide ring that comprises the AP-1 modulating sequence. The intramolecular bond may be a backbone to backbone, side-chain to backbone or side-chain to side-chain bond (i.e., terminal functional groups of a linear peptide and/or side chain functional groups of a terminal or interior residue may be linked to achieve cyclization). Preferred intramolecular bonds include, but are not limited to, disulfide bonds; amide bonds between terminal functional groups, between residue side chains or between one terminal functional group and one residue side chain; thioether bonds and (δ₁, δ₁′)-ditryptophan or a derivative thereof. Preferred cyclic peptide modulating agents generally comprise at least eight residues, and more preferably between 10 and 15 residues, within the cyclic peptide ring.

As noted above, modulating agents may comprise polypeptides or salts thereof, containing only amino acid residues linked by peptide bonds, or may additionally contain non-peptide regions, such as linkers. Peptide regions of a modulating agent may comprise residues of L-amino acids, D-amino acids, or any combination thereof. Amino acids may be from natural or non-natural sources; α- and β-amino acids are generally preferred. The 20 L-amino acids commonly found in proteins are identified herein by the conventional three-letter or one-letter abbreviations.

A modulating agent may also contain rare amino acids (such as 4-hydroxyproline or hydroxylysine), organic acids or amides and/or derivatives of common amino acids, such as amino acids having the C-terminal carboxylate esterified (e.g., benzyl, methyl or ethyl ester) or amidated and/or having modifications of the N-terminal amino group (e.g., acetylation or alkoxycarbonylation), with or without any of a wide variety of side-chain modifications and/or substitutions (e.g., methylation, benzylation, t-butylation, tosylation, alkoxycarbonylation) and the like). Preferred derivatives include amino acids having a C-terminal amide group. Residues other than common amino acids that may be present with a modulating agent include, but are not limited to, 2-mercaptoaniline, 2-mercaptoproline, ornithine, diaminobutyric acid, α-aminoadipic acid, m-aminomethylbenzoic acid and α, β-diaminopropionic acid.

As noted above, a modulating agent may comprise a peptide analogue or a peptidomimetic of a naturally-occurring AP-1 modulating amino acid sequence from NIC-1, provided that the analogue or peptidomimetic retains the ability to inhibit an AP-1 mediated response. In general, a peptide analogue may contain conservative substitutions such that the ability to modulate an AP-1 mediated response is not substantially diminished. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. The critical determining feature of a peptide analogue is the ability to modulate an AP-1 mediated response. Such an ability may be evaluated using assays substantially similar to those provided in the Example section below.

Peptide analogs may further be identified by performing mutational analysis of the RAM domain of NIC-1 and assaying resulting mutants for AP-1 modulating ability. For example, alanine scanning mutagenesis may be performed to identify key residues necessary for modulating activities. Specifically, the amino acid sequence of the RAM domain, provided in SEQ ID NO: 1, may be submitted to alanine scanning in order to discern the effect of each residue on the ability of the peptide to modulate AP-1 mediated transcription. Alanine scanning mutagenesis generates a small and systematic set of mutant peptides whose inhibitory activity can be readily tested using the assay techniques set forth herein. Alanine substitution does not impose new structural effects related to hydrogen bonding, unusual hydrophobicity, or steric bulk, and it is expected to cause minimal perturbation of secondary structure; alanine is compatible with all secondary structures in both buried and solvent-exposed positions (Abroi et al., i J. Virology, 70(9):6169, 1996; Cunningham et al., Science, 244: 1081, 1989; Rose et al., Science, 229:834, 1985; Klapper et al., Biochem Biophys Res Communic, 78(3):1018, 1977; Chothia et al., J. Molecu Biol, 105(1):1, 1976). Also, in contrast to amino acid deletions, substitution with alanine preserves the original spacing of residues. Thus, alanine scanning is an exemplary technique for isolating the effect of particular amino acids within the context of the NIC-1 or RAM sequences. Based upon such an analysis, one of skill in the art could prepare peptide analogs which exhibit AP-1 modulating activity in similar fashion to the naturally-occurring polypeptide sequences.

A peptidomimetic according to the present invention is a compound that is structurally similar to a NIC-1 derived polypeptide, such that the peptidomimetic retains the ability to modulate an AP-1 mediated response. In general, peptidomimetics are organic compounds that mimic the three-dimensional shape and activity of a particular polypeptide. It is now accepted that peptidomimetics may be designed based on techniques that evaluate three dimensional shape, such as nuclear magnetic resonance (NMR) and computational techniques. NMR is widely used for structural analysis of molecules. Cross-peak intensities in Nuclear Overhauser Enhancement (NOE) spectra, coupling constants and chemical shifts depend on the conformation of a compound. NOE data provide the inter-proton distance between protons through space. This information may be used to facilitate calculation of the lowest energy conformation for the relevant peptide sequence. Once the lowest energy conformation is known, the three-dimensional shape to be mimicked is known. It should be understood that, within embodiments described herein, a peptidomimetic (and analog) may be substituted for the amino acid sequence of the polypeptide on which the peptidomimetic is based.

Examples of peptidomimetics encompassed by the present invention include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. In addition to rational designing, as described above, a peptidomimetic can be obtained by, for example, screening libraries of natural and synthetic compounds for compounds capable of modulating AP-1 transactivation.

Peptide-based modulating agents (or peptide portions of modulating agents) as described herein may be synthesized by methods well known in the art, including chemical synthesis and recombinant DNA methods. For modulating agents up to about 50 residues in length, chemical synthesis may be performed using solution phase or solid phase peptide synthesis techniques, in which a peptide linkage occurs through the direct condensation of the α-amino group of one amino acid with the a-carboxy group of the other amino acid with the elimination of a water molecule. Peptide bond synthesis by direct condensation, as formulated above, requires suppression of the reactive character of the amino group of the first and of the carboxyl group of the second amino acid. The masking substituents must permit their ready removal, without inducing breakdown of the labile peptide molecule.

In solution phase synthesis, a wide variety of coupling methods and protecting groups may be used (see Gross and Meienhofer, eds., “The Peptides: Analysis, Synthesis, Biology,” Vol. 1-4 (Academic Press, 1979); Bodansky and Bodansky, “The Practice of Peptide Synthesis,” 2d ed. (Springer Verlag, 1994)). In addition, intermediate purification and linear scale up are possible. Those of ordinary skill in the art will appreciate that solution synthesis requires consideration of main chain and side chain protecting groups and activation method. In addition, careful segment selection is necessary to minimize racemization during segment condensation. Solubility considerations are also a factor.

Solid phase peptide synthesis uses an insoluble polymer for support during organic synthesis. The polymer-supported peptide chain permits the use of simple washing and filtration steps instead of laborious purifications at intermediate steps. Solid-phase peptide synthesis may generally be performed according to the method of Merrifield et al., J. Am. Chem. Soc. 85:2149, 1963, which involves assembling a linear peptide chain on a resin support using protected amino acids. Solid phase peptide synthesis typically utilizes either the Boc or Fmoc strategy. The Boc strategy uses a 1% cross-linked polystyrene resin. The standard protecting group for α-amino functions is the tert-butyloxycarbonyl (Boc) group. This group can be removed with dilute solutions of strong acids such as 25% trifluoroacetic acid (TFA). The next Boc-amino acid is typically coupled to the amino acyl resin using dicyclohexylcarbodiimide (DCC). Following completion of the assembly, the peptide-resin is treated with anhydrous HF to cleave the benzyl ester link and liberate the free peptide. Side-chain functional groups are usually blocked during synthesis by benzyl-derived blocking groups, which are also cleaved by HF. The free peptide is then extracted from the resin with a suitable solvent, purified and characterized. Newly synthesized peptides can be purified, for example, by gel filtration, HPLC, partition chromatography and/or ion-exchange chromatography, and may be characterized by, for example, mass spectrometry or amino acid sequence analysis. In the Boc strategy, C-terminal amidated peptides can be obtained using benzhydrylamine or methylbenzhydrylamine resins, which yield peptide amides directly upon cleavage with HF.

In the procedures discussed above, the selectivity of the side-chain blocking groups and of the peptide-resin link depends upon the differences in the rate of acidolytic cleavage. Orthogonal systems have been introduced in which the side-chain blocking groups and the peptide-resin link are completely stable to the reagent used to remove the α-protecting group at each step of the synthesis. The most common of these methods involves the 9-fluorenylmethyloxycarbonyl (Fmoc) approach. Within this method, the side-chain protecting groups and the peptide-resin link are completely stable to the secondary amines used for cleaving the N-α-Fmoc group. The side-chain protection and the peptide-resin link are cleaved by mild acidolysis. The repeated contact with base makes the Merrifield resin unsuitable for Fmoc chemistry, and β-alkoxybenzyl esters linked to the resin are generally used. Deprotection and cleavage are generally accomplished using TFA.

Those of ordinary skill in the art will recognize that, in solid phase synthesis, deprotection and coupling reactions must go to completion and the side-chain blocking groups must be stable throughout the entire synthesis. In addition, solid phase synthesis is generally most suitable when peptides are to be made on a small scale.

Acetylation of the N-terminus can be accomplished by reacting the final peptide with acetic anhydride before cleavage from the resin. C-amidation may be accomplished using an appropriate resin such as methylbenzhydrylamine resin using the Boc technology.

Following synthesis of a linear peptide, cyclization may be achieved if desired by any of a variety of techniques well known in the art. Within one embodiment, a bond may be generated between reactive amino acid side chains. For example, a disulfide bridge may be formed from a linear peptide comprising two thiol-containing residues by oxidizing the peptide using any of a variety of methods. Within one such method, air oxidation of thiols can generate disulfide linkages over a period of several days using either basic or neutral aqueous media. The peptide is used in high dilution to minimize aggregation and intermolecular side reactions. This method suffers from the disadvantage of being slow but has the advantage of only producing H₂O as a side product. Alternatively, strong oxidizing agents such as I₂ and K₃ Fe(CN)₆ can be used to form disulfide linkages. Those of ordinary skill in the art will recognize that care must be taken not to oxidize the sensitive side chains of Met, Tyr, Trp or His. Cyclic peptides produced by this method require purification using standard techniques, but this oxidation is applicable at acid pHs. Oxidizing agents also allow concurrent deprotection/oxidation of suitable S-protected linear precursors to avoid premature, nonspecific oxidation of free cysteine.

DMSO, unlike I₂ and K₃ Fe(CN)₆, is a mild oxidizing agent which does not cause oxidative side reactions of the nucleophilic amino acids mentioned above. DMSO is miscible with H₂O at all concentrations, and oxidations can be performed at acidic to neutral pHs with harmless byproducts. Methyltrichlorosilane-diphenylsulfoxide may alternatively be used as an oxidizing agent, for concurrent deprotection/oxidation of S-Acm, S-Tacm or S-t-Bu of cysteine without affecting other nucleophilic amino acids. There are no polymeric products resulting from intermolecular disulfide bond formation. Suitable thiol-containing residues for use in such oxidation methods include, but are not limited to, cysteine, β,β-dimethyl cysteine (penicillamine or Pen), β,β-tetramethylene cysteine (Tmc), β,β-pentamethylene cysteine (Pmc), β-mercaptopropionic aid (Mpr), β, β-pentamethylene-.beta.-mercaptopropionic acid (Pmp), 2-mercaptobenzene, 2-mercaptoaniline and 2-mercaptoproline. Within another embodiment, cyclization may be achieved by amide bond formation. For example, a peptide bond may be formed between terminal functional groups (i.e., the amino and carboxy termini of a linear peptide prior to cyclization), with or without an N-terminal acetyl group and/or a C-terminal amide. Within another such embodiment, the linear peptide comprises a D-amino acid. Alternatively, cyclization may be accomplished by linking one terminus and a residue side chain or using two side chains, with or without an N-terminal acetyl group and/or a C-terminal amide. Residues capable of forming a lactam bond include lysine, ornithine (Orn), α-amino adipic acid, m-aminomethylbenzoic acid, α,β-diaminopropionic acid, glutamate or aspartate.

Methods for forming amide bonds are well known in the art and are based on well established principles of chemical reactivity. Within one such method, carbodiimide-mediated lactam formation can be accomplished by reaction of the carboxylic acid with DCC, DIC, EDAC or DCCI, resulting in the formation of an O-acylurea that can be reacted immediately with the free amino group to complete the cyclization. The formation of the inactive N-acylurea, resulting from O to N migration, can be circumvented by converting the O-acylurea to an active ester by reaction with an N-hydroxy compound such as 1-hydroxybenzotriazole, 1-hydroxysuccinimide, 1-hydroxynorbornene carboxamide or ethyl 2-hydroximino-2-cyanoacetate. In addition to minimizing O to N migration. These additives also serve as catalysts during cyclization and assist in lowering racemization. Alternatively, cyclization can be performed using the azide method, in which a reactive azide intermediate is generated from an alkyl ester via a hydrazide. Hydrazinolysis of the terminal ester necessitates the use of a t-butyl group for the protection of side chain carboxyl functions in the acylating component. This limitation can be overcome by using diphenylphosphoryl acid (DPPA), which furnishes an azide directly upon reaction with a carboxyl group. The slow reactivity of azides and the formation of isocyanates by their disproportionation restrict the usefulness of this method. The mixed anhydride method of lactam formation is widely used because of the facile removal of reaction by-products. The anhydride is formed upon reaction of the carboxylate anion with an alkyl chloroformate or pivaloyl chloride. The attack of the amino component is then guided to the carbonyl carbon of the acylating component by the electron donating effect of the alkoxy group or by the steric bulk of the pivaloyl chloride t-butyl group, which obstructs attack on the wrong carbonyl group. Mixed anhydrides with phosphoric acid derivatives have also been successfully used. Alternatively, cyclization can be accomplished using activated esters. The presence of electron withdrawing substituents on the alkoxy carbon of esters increases their susceptibility to aminolysis. The high reactivity of esters of p-nitrophenol, N-hydroxy compounds and polyhalogenated phenols has made these “active esters” useful in the synthesis of amide bonds. The last few years have witnessed the development of benzotriazolyloxytris-(dimethylamino)phosphonium hexafluorophosphonate (BOP) and its congeners as advantageous coupling reagents. Their performance is generally superior to that of the well established carbodiimide amide bond formation reactions.

Within a further embodiment, a thioether linkage may be formed between the side chain of a thiol-containing residue and an appropriately derivatized α-amino acid. By way of example, a lysine side chain can be coupled to bromoacetic acid through the carbodiimide coupling method (DCC, EDAC) and then reacted with the side chain of any of the thiol containing residues mentioned above to form a thioether linkage. In order to form dithioethers, any two thiol containing side-chains can be reacted with dibromoethane and diisopropylamine in DMF. Cyclization may also be achieved using δ₁,δ₁′-Ditryptophan.

For longer peptide-containing modulating agents, recombinant methods are preferred for synthesis. Within such methods, all or part of a modulating agent can be synthesized in living cells, using any of a variety of expression vectors known to those of ordinary skill in the art to be appropriate for the particular host cell. Suitable host cells may include bacteria, yeast cells, mammalian cells, insect cells, plant cells, algae and other animal cells (e.g., hybridoma, CHO, myeloma). The DNA sequences expressed in this manner may encode AP-1 modulating portions of Notch-1 and/or other sequences including internalization signals (cytoplasmic and nuclear). AP-1 modulating sequences may be prepared based on known cDNA or genomic sequences which may be isolated by screening an appropriate library with probes designed based on such known sequences. Notch sequences are known from a variety of organisms including the human Notch-1 coding sequence deposited with GenBank (Accession No. AF308602). Screens may generally be performed as described in Sambrook et al., Molecular Cloning. A Laboratory Manual, Cold Spring Harbor Laboratories, Cold Spring Harbor, N.Y., 1989 (and references cited therein). Polymerase chain reaction (PCR) may also be employed, using oligonucleotide primers in methods well known in the art, to isolate nucleic acid molecules encoding all or a portion of an endogenous Notch-1.

The invention further contemplates a method of generating sets of combinatorial libraries of a defined AP-1 modulating polypeptide isolated from NIC-1. This approach is especially useful for identifying potential variant sequences (e.g. homologs) that are functional in modulating AP-1 mediated transactivation. Combinatorially-derived homologs can be generated which have, e.g., greater affinity, a enhanced potency relative to native Notch-1 peptide sequences, or intracellular half-lives different than the corresponding wild-type Notch-1 peptide. For example, the altered peptide can be rendered either more stable or less stable to proteolytic degradation or other cellular process which result in destruction of, or otherwise inactivation of, the peptide. Such homologs can be utilized to alter the envelope of therapeutic application by modulating the half-life of the peptide. For instance, a short half-life can give rise to more transient biological effects and can allow tighter control of peptide levels within the cell.

In one embodiment, a NIC-1 based peptide library can be derived by combinatorial chemistry, such as by techniques which are available in the art for generating combinatorial libraries of small organic/peptide libraries. See, for example, Blondelle et al. (1995) Trends Anal. Chem. 14:83; the Affymax U.S. Pat. Nos. 5,359,115 and 5,362,899; the Ellman U.S. Pat. No. 5,288,514; the Still et al. PCT publication WO 94/08051; Chen et al. (1994) JACS 116:2661; Kerr et al. (1993) JACS 115:252; PCT publications WO092/10092, WO93/09668 and WO91/07087; and the Lerner et al. PCT publication WO93/20242).

The combinatorial peptide library may be produced by way of a degenerate library of genes encoding a library of polypeptides which each include at least a portion of NIC-1 sequences. For instance, a mixture of synthetic oligonucleotides can be enzymatically ligated into gene sequences such that the degenerate set of NIC-1 nucleotide sequences are expressible as individual polypeptides, or alternatively, as a set of larger fusion proteins (e.g. for phage display) containing the set of NIC-1-based peptide sequences therein.

There are many ways by which the gene library of potential NIC-1 homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential sequences. The synthesis of degenerate oligonucleotides is well known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp. 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

A wide range of techniques are known in the art for screening gene products of combinatorial libraries made by techniques provided above. Such techniques will be generally adaptable for rapid screening of the gene libraries generated by the combinatorial mutagenesis of NIC-1 derived sequences. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Such illustrative assays are amenable to high throughput analysis as necessary to screen large numbers of degenerate sequences created by combinatorial mutagenesis techniques. Specifically, an AP-1 responsive reporter construct, as provided in the Example section below, may be used to rapidly screen large numbers of potential homologs for AP-1 modulating ability.

A modulating agent according to the present invention comprises an internalization moiety. An internalization moiety is any moiety (such as a polypeptide, liposome or particle) that can be used to improve the ability of an agent to penetrate the lipid bilayer of the cellular plasma membrane, thus enabling the agent to readily enter the cytoplasm. In addition, an internalization moiety may also refer to a moiety capable of directing the modulating agent into the nuclear compartment. An internalization moiety may be linked via covalent attachment or a non-covalent interaction mediated by, for example, ionic bonds, hydrogen bonds, van der waals forces and/or hydrophobic interactions, such that the internalization moiety and modulating agent remain in close proximity under physiological conditions.

Within certain embodiments, an internalization moiety is a peptide internalization sequence capable of facilitating entry of the modulating agent into the cytosol of a living cell. One suitable internalization sequence is a 16 amino acid peptide derived from the third helix of the Antennapedia protein, and having the sequence RQIKIWFQNRRMKWKK (see Prochiantz, Curr. Op. Neurobiol. 6:629-34, 1996) or RQIKIWPQNRRNKWKK. Analogues of this sequence (i.e., sequences having at least 25% sequence identity, such that the ability to facilitate entry into the cytosol is not diminished) may also be employed. One such analogue is therefor KKWKKWWKKWWKKWKK.

Alternatively, an internalization sequence may be unrelated to the Antennapedia sequence. Any sequence that facilitates entry to the cell, via a cell surface receptor or other means, may be employed. Protein-derived helical peptide sequences that may be used as internalization sequences include, but are not limited to, KLALKLALKLAKAALKLA see Oehlke et al., Biochim. Biophys. Acta 1414:127-139, 1998, and references cited therein). Other internalization sequences include the 11 amino acid TAT protein transduction domain YGRKKRRQRRR; see Nagahara et al., Nature Medicine 4:1449-1452, 1998) and the transduction domain of HSV VP22 (see Elliot and O'Hare, Cell 88:223-244, 1997).

In general, the ability of a sequence to facilitate entry into the cytosol may be evaluated in any of a variety of ways. For example, a candidate internalization sequence may be covalently linked to the AP-1 modulating portion of the agent and contacted with cells. The ability of such a construct to modulate an AP-1 mediated response, as described herein, may then be assessed. Alternatively, the ability of a candidate internalization sequence to cross the plasma membrane may be assessed directly using any assay known in the art. Within such any assay, an internalization sequence should result in a response that is statistically greater than that observed in the absence of internalization sequence.

While not wishing to be bound by any particular theory, it is noted that hydrophilic polypeptides may be also be physiologically transported across the membrane barriers by coupling or conjugating the polypeptide to a transportable peptide which is capable of crossing the membrane by receptor-mediated transcytosis. Suitable internalizing peptides of this type can be generated using all or a portion of, e.g., a histone, insulin, transferrin, basic albumin, prolactin and insulin-like growth factor I (IGF-I), insulin-like growth factor II (IGF-II) or other growth factors. For instance, it has been found that an insulin fragment, showing affinity for the insulin receptor on capillary cells, and being less effective than insulin in blood sugar reduction, is capable of transmembrane transport by receptor-mediated transcytosis and can therefor serve as an internalizing peptide for the subject transcellular peptides and peptidomimetics. Preferred growth factor-derived internalizing peptides include EGF (epidermal growth factor)-derived peptides, such as CMHIESLDSYTC and CMYIEALDKYAC; TGF-beta (transforming growth factor beta)-derived peptides; peptides derived from PDGF (platelet-derived growth factor) or PDGF-2; peptides derived from IGF-I (insulin-like growth factor) or IGF-II; and FGF (fibroblast growth factor)-derived peptides.

Another class of translocating/internalizing peptides exhibits pH-dependent membrane binding. For an internalizing peptide that assumes a helical conformation at an acidic pH, the internalizing peptide acquires the property of amphiphilicity, e.g., it has both hydrophobic and hydrophilic interfaces. In one embodiment, within a pH range of approximately 5.0-5.5, an internalizing peptide forms an α-helical, amphiphilic structure that facilitates insertion of the moiety into a target membrane. An α-helix-inducing acidic pH environment may be found, for example, in the low pH environment present within cellular endosomes. Such internalizing peptides can be used to facilitate transport of AP-1 modulating peptides and peptidomimetics, taken up by an endocytic mechanism, from endosomal compartments to the cytoplasm. A preferred pH-dependent membrane-binding internalizing peptide includes a high percentage of helix-forming residues, such as glutamate, methionine, alanine and leucine. In addition, a preferred internalizing peptide sequence includes ionizable residues having pKa's within the range of pH 5-7, so that a sufficient uncharged membrane-binding domain will be present within the peptide at pH 5 to allow insertion into the target cell membrane.

Yet other preferred internalizing peptides include peptides of apo-lipoprotein A-1 and B; peptide toxins, such as melittin, bombolittin, delta hemolysin and the pardaxins; antibiotic peptides, such as alamethicin; peptide hormones, such as calcitonin, corticotrophin releasing factor, beta endorphin, glucagon, parathyroid hormone, pancreatic polypeptide; and peptides corresponding to signal sequences of numerous secreted proteins. In addition, exemplary internalizing peptides may be modified through attachment of substituents that enhance the α-helical character of the internalizing peptide at acidic pH.

Yet another class of internalizing peptides suitable for use within the present invention include hydrophobic domains that are “hidden” at physiological pH, but are exposed in the low pH environment of the target cell endosome. Upon pH-induced unfolding and exposure of the hydrophobic domain, the moiety binds to lipid bilayers and effects translocation of the covalently linked polypeptide into the cell cytoplasm. Such internalizing peptides may be modeled after sequences identified in, e.g., Pseudomonas exotoxin A, clathrin, or Diphtheria toxin.

Pore-forming proteins or peptides may also serve as internalizing peptides herein. Pore-forming proteins or peptides may be obtained or derived from, for example, C9 complement protein, cytolytic T-cell molecules or NK-cell molecules. These moieties are capable of forming ring-like structures in membranes, thereby allowing transport of attached polypeptide through the membrane and into the cell interior.

In preferred embodiments, it is desirable to include a nuclear localization signal as part of the modulating agent. It is conceivable that modulating agents will have both an internalization moiety for localization to the cytoplasm as well as a moiety directing nuclear localization. Naturally-occurring NIC-1 has at least two putative nuclear localization signals and these sequences may be included in addition to AP-1 modulating sequences so that the modulating agent may gain entry to the nucleoplasm. Data provided in the Examples section below suggests that nuclear localization is important for the AP-1 modulating activity of NIC-1 to manifest. Thus, preferred embodiments of modulating agents according to the present invention will include nuclear localization signals from NIC-1 or another source, as described below, to direct modulating agents into the nucleoplasm.

In general, the nuclear localization signal used in the present invention is not particularly limited as long as it has the activity to translocate a substance to which the signal sequence is attached into the nucleus. Amino acid sequences have been determined for the nuclear localization signals of a variety of proteins found in the cells of vertebrates. For example, in the case of translocating modulating agents according to the present invention into the nucleus, it is possible to use the nuclear localization signal of SV40 VP 1, SV40 large T antigen, or hepatitis D virus delta. antigen, or a sequence containing “PKKKRKV” which represents the minimum unit having the nuclear translocation activity within the nuclear localization signal of SV40 large T antigen. The NLS within the amino acid sequence of the SV40 (monkey virus) large T antigen was reported by Kalderon, et al., Cell, 39:499-509, 1984 (see SEQ.1). This “single basic domain” SV40 NLS is considered the canonical prototype signal to which all others have been compared (Kalderon, 1984, supra; Forbes, Annu. Rev. Cell Biota., 8:495-527, 1992), and many NLSs resemble it. Many other NLSs, however, more closely resemble the first identified NLS, that of the Xenopus (African clawed toad) protein, nucleoplasmin. This “double basic domain” NLS was initially defined by protease digestion of nucleoplasmin by Dingwall, et al., Cell, 30:449-458, 1982 (also see Dingwall, et al., J. Cell Biol., 107:841-849; 1988).

Experiments have been performed in a number of laboratories in which the effects of NLSs incorporated in synthetic peptides or grafted onto reporter proteins not normally targeted to the cell nucleus have been studied. Localization studies revealed that the NLSs cause these peptides and reporter proteins to be concentrated in the nucleus. See, for example, Dingwall, and Laskey, Ann. Rev. Cell Biol., 2:367-390, 1986; Bonnerot, et al., Proc. Natl. Acad. Sci. USA, 84:6795-6799, 1987; Galileo, et al., Proc. Natl. Acad. Sci. USA, 87:458-462, 1990; Forbes, 1992, supra.

In the generation of modulating agents including AP-1 modulating peptides described herein, it may be necessary to include unstructured linkers in order to ensure proper folding of the various peptide domains, and prevent steric or other interference of the respective molecule. Many synthetic and natural linkers are known in the art and can be adapted for use in the present invention. In general, spacers may be amino acid residues (e.g., amino hexanoic acid) or peptides, or may be other bi- or multi-functional compounds that can be covalently linked to at least two peptide sequences. Covalent linkage may be achieved via direct condensation or other well known techniques.

According to one aspect of this invention, modulating agents according to the present invention may be administered directly to target cells. Direct delivery of such therapeutics may be facilitated by formulation of the composition in any pharmaceutically acceptable dosage form, e.g., for delivery orally, intratumorally, peritumorally, interlesionally, intravenously, intramuscularly, subcutaneously, periolesionally, or topical routes, to exert local therapeutic effects.

Topical administration of the therapeutic is advantageous since it allows localized concentration at the site of administration with minimal systemic adsorption. This simplifies the delivery strategy of the agent to the disease site and reduces the extent of toxicological characterization. Furthermore, the amount of material to be applied is far less than that required for other administration routes.

In one embodiment, the membrane barrier can be overcome by utilizing an internalization moiety comprising lipid formulations closely resembling the lipid composition of natural cell membranes. In particular, the subject peptides, analogs, or peptidomimetics are encapsulated in liposomes to form pharmaceutical preparations suitable for administration to living cells. The Yarosh U.S. Pat. No. 5,190,762 demonstrates that proteins can be delivered across the outer skin layer and into living cells, without receptor binding, by liposome encapsulation. These lipids are able to fuse with the cell membranes on contact, and in the process, the associated peptides, analogs, or peptidomimetics are delivered intracellularly. Lipid complexes can not only facilitate intracellular transfers by fusing with cell membranes but also by overcoming charge repulsions between the cell membrane and the molecule to be inserted. The lipids of the formulations comprise an amphipathic lipid, such as the phospholipids of cell membranes, and form hollow lipid vesicles, or liposomes, in aqueous systems. This property can be used to entrap peptides, analogs, or peptidomimetics within the liposomes.

Liposomes offer several advantages. They are non-toxic and biodegradable in composition; they display long circulation half-lives; and recognition molecules can be readily attached to their surface for targeting to tissues. Finally, cost effective manufacture of liposome-based pharmaceuticals, either in a liquid suspension or lyophilized product, has demonstrated the viability of this technology as an acceptable drug delivery system.

Liposomes have been described in the art as in vivo delivery vehicles. The structure of various types of lipid aggregates varies, depending on composition and method of forming the aggregate. Such aggregates include liposomes, unilamellar vesicles, multilamellar vesicles, micelles and the like, having particle sizes in the nanometer to micrometer range. Methods of making lipid aggregates are by now well-known in the art. For example, the liposomes may be made from natural and synthetic phospholipids, glycolipids, and other lipids and lipid congeners; cholesterol, cholesterol derivatives and other cholesterol congeners; charged species which impart a net charge to the membrane; reactive species which can react after liposome formation to link additional molecules to the liposome membrane; and other lipid soluble compounds which have chemical or biological activity.

In another embodiment, the present invention relates to gene therapy constructs containing a nucleic acid encoding an AP-1 modulating peptide of the present invention, operably linked to at least one transcriptional regulatory sequence. Such constructs preferably encode a nuclear localization signal, either from native NIC-1 or from another source, which acts to direct the AP-1 modulating peptide to the nuclear compartment. The gene constructs of the present invention are formulated to be used as a part of a gene therapy protocol to deliver the subject therapeutic protein to a target cell in an animal.

Any of the methods known to the art for the insertion of DNA fragments into a vector may be used to construct expression vectors consisting of appropriate transcriptional/translational control signals and the desired NIC-1 peptide-encoding nucleotide sequence. See, for example, Maniatis T., Fritsch E. F., and Sambrook J. (1989): Molecular Cloning (A Laboratory Manual), Cold Spring Harbor Laboratory, Cold Spring Harbor, N.Y.; and Ausubel F. M., Brent R., Kingston R. E., Moore, D. D., Seidman J. G., Smith J. A., and Struhl K. (1992): Current Protocols in Molecular Biology, John Wiley & Sons, New York. These methods may include in vitro DNA recombinant and synthetic techniques and in vivo genetic recombination. Expression of a nucleic acid sequence encoding an a peptide may be regulated by a second nucleic acid sequence so that the peptide is expressed in a host infected or transfected with the recombinant DNA molecule. For example, expression of a Notch-1 peptide may be controlled by any promoter/enhancer element known in the art. The promoter activation may be tissue specific or inducible by a metabolic product or administered substance.

Promoters/enhancers which may be used to control the expression of the Notch-1 peptide in vivo include, but are not limited to, the native Notch-1 promoter, the cytomegalovirus (CMV) promoter/enhancer (Karasuyama et al., 1989, J. Exp. Med., 169:13), the human β-actin promoter (Gunning et al. (1987) PNAS 84:4831-4835), the glucocorticoid-inducible promoter present in the mouse mammary tumor virus long terminal repeat (MMTV LTR) (Klessig et al. (1984) Mol. Cell Biol. 4:1354-1362), the long terminal repeat sequences of Moloney murine leukemia virus (MuLV LTR) (Weiss et al. (1985) RNA Tumor Viruses, Cold Spring Harbor Laboratory, Cold Spring Harbor, N.Y.), the SV40 early or late region promoter (Bernoist et al. (1981) Nature 290:304-310; Templeton et al. (1984) Mol. Cell Biol, 4:817; and Sprague et al. (1983) J. Virol., 45:773), the promoter contained in the 3′ long terminal repeat of Rous sarcoma virus (RSV) (Yamamoto et al., 1980, Cell, 22:787-797), the herpes simplex virus (HSV) thymidine kinase promoter/enhancer (Wagner et al. (1981) PNAS 82:3567-71), and the herpes simplex virus LAT promoter (Wolfe et al. (1992) Nature Genetics, 1:379-384), and Keratin gene promoters, such as Keratin 14.

Expression constructs of the subject Notch-1 peptides may be administered in any biologically effective carrier, e.g. any formulation or composition capable of effectively delivering the recombinant gene to cells in vivo. Approaches include insertion of the Notch-1 peptide coding sequence in viral vectors including recombinant retroviruses, adenovirus, adeno-associated virus, and herpes simplex virus-1, or recombinant eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation carried out in vivo. It will be appreciated that because transduction of appropriate target cells represents the critical first step in gene therapy, choice of the particular gene delivery system will depend on such factors as the phenotype of the intended target and the route of administration, e.g. locally or systemically.

A preferred approach for in vivo introduction of nucleic acid into a cell is by use of a viral vector containing nucleic acid encoding the particular Notch-1 peptide possessing AP-1 modulating activity. Infection of cells with a viral vector has the advantage that a large proportion of the targeted cells can receive the nucleic acid. Additionally, molecules encoded within the viral vector, e.g., the recombinant Notch-1 peptide, are expressed efficiently in cells which have taken up viral vector nucleic acid.

In addition to viral transfer methods, such as those illustrated above, non-viral methods can also be employed to cause expression of a Notch-1 peptide in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the construct encoding the NIC-1 polypeptides by the targeted cell. Exemplary gene delivery systems of this type include liposomal derived systems, poly-lysine lysine conjugates, and artificial viral envelopes.

In clinical settings, the gene delivery systems for the therapeutic Notch-1 peptide coding sequence can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or “gene gun” techniques. In preferred embodiments, the gene therapy construct of the present invention is applied topically to target cells of the skin or mucosal tissue. A NIC-1 peptide gene construct can, in one embodiment, be delivered in a gene therapy construct by electroporation using techniques described, for example, by Dev et al. ((1994) Cancer Treat Rev 20:105-115).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced intact from recombinant cells, e.g. retroviral vectors, the pharmaceutical preparation can comprise one or more cells which produce the gene delivery system.

As noted above, modulating agents are capable of modulating AP-1 mediated gene transcription. This ability may generally be evaluated using any suitable assay known to those of ordinary skill in the art to directly evaluate AP-1 mediated gene transcription (e.g., using amplification or hybridization techniques to evaluate the level of mRNA corresponding to a gene that is transcribed in response to AP-1). Alternatively, the effect of a modulating agent on a response associated with AP-1 mediated gene transcription may be measured. A representative method of assaying AP-1 activity is provided herein in which a reporter gene, in this case luciferase, is fused to an AP-1 responsive promoter. Effects of a particular modulating agent or AP-1 transactivation may be assessed by contacting cells transformed with the reporter construct with the potential modulating agent and assaying the luciferase activity against appropriate controls. Modulating agents are identified as those agents capable of providing a statistically meaningful difference in AP-1 mediated transactivation in comparison to controls.

A modulating agent according to the present invention may, but need not, be linked to one or more additional molecules. Although modulating agents as described herein may preferentially bind to specific tissues or cells, and thus may be sufficient to target a desired site in vivo, it may be beneficial for certain applications to include an additional targeting agent. Accordingly, a targeting agent may be associated with a modulating agent to facilitate targeting to one or more specific tissues. As used herein, a “targeting agent” may be any substance (such as a compound or cell) that, when associated with a modulating agent enhances the transport of the modulating agent to a target tissue, thereby increasing the local concentration of the modulating agent.

Targeting agents include antibodies or fragments thereof, receptors, ligands and other molecules that bind to cells of, or in the vicinity of, the target tissue. Known targeting agents include serum hormones, antibodies against cell surface antigens, lectins, adhesion molecules, tumor cell surface binding ligands, steroids, cholesterol, lymphokines, fibrinolytic enzymes and those drugs and proteins that bind to a desired target site. Among the many monoclonal antibodies that may serve as targeting agents are anti-TAC, or other interleukin-2 receptor antibodies; 9.2.27 and NR-ML-05, reactive with the 250 kilodalton human melanoma-associated proteoglycan; and NR-LU-10, reactive with a pancarcinoma glycoprotein. An antibody targeting agent may be an intact (whole) molecule, a fragment thereof, or a functional equivalent thereof. Examples of antibody fragments are F(ab′)2, -Fab′, Fab and F[v] fragments, which may be produced by conventional methods or by genetic or protein engineering. Linkage is generally covalent and may be achieved by, for example, direct condensation or other reactions, or by way of bi- or multi-functional linkers. Within other embodiments, it may also be possible to target a polynucleotide encoding a modulating agent to a target tissue, thereby increasing the local concentration of modulating agent. Such targeting may be achieved using well known techniques, including retroviral and adenoviral infection, as described above.

Within certain aspects of the present invention, one or more modulating agents as described herein may be present within a pharmaceutical composition. A pharmaceutical composition comprises one or more modulating agents in combination with one or more pharmaceutically or physiologically acceptable carriers, diluents or excipients. Such compositions may comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Within yet other embodiments, compositions of the present invention may be formulated as a lyophilizate. One or more modulating agents (alone or in combination with a targeting agent and/or drug) may, but need not, be encapsulated within liposomes using well known technology. Compositions of the present invention may be formulated for any appropriate manner of administration, including for example, topical, oral, nasal, intravenous, intracranial, intraperitoneal, subcutaneous, or intramuscular administration.

A pharmaceutical composition may also, or alternatively, contain one or more drugs, which may be linked to a modulating agent or may be free within the composition. Virtually any drug may be administered in combination with a modulating agent as described herein, for a variety of purposes as described below. Examples of types of drugs that may be administered with a modulating agent include analgesics, anesthetics, antianginals, antifungals, antibiotics, anticancer drugs (e.g., taxol or mitomycin C), antiinflammatories (e,g, ibuprofen and indomethacin), antihelmintics, antidepressants, antidotes, antiemetics, antihistamines, antihypertensives, antimalarials, antimicrotubule agents (e.g., colchicine or vinca alkaloids), antimigraine agents, antimicrobials, antiphsychotics, antipyretics, antiseptics, anti-signaling agents (e.g., protein kinase C inhibitors or inhibitors of intracellular calcium mobilization), antiarthritics, antithrombin agents, antituberculotics, antitussives, antivirals, appetite suppressants, cardioactive drugs, chemical dependency drugs, cathartics, chemotherapeutic agents, coronary, cerebral or peripheral vasodilators, contraceptive agents, depressants, diuretics, expectorants, growth factors, hormonal agents, hypnotics, immunosuppression agents, narcotic antagonists, parasympathomimetics, sedatives, stimulants, sympathomimetics, toxins (e.g., cholera toxin), tranquilizers and urinary antiinfectives.

The compositions described herein may be administered as part of a sustained release formulation (i.e., a formulation such as a capsule or sponge that effects. a slow release of modulating agent following administration). Such formulations may generally be prepared using well known technology and administered by, for example, oral, rectal or subcutaneous implantation, or by implantation at the desired target site. Sustained-release formulations may contain a modulating agent dispersed in a carrier matrix and/or contained within a reservoir surrounded by a rate controlling membrane (see, e.g., European Patent Application 710,491 A). Carriers for use within such formulations are biocompatible, and may also be biodegradable; preferably the formulation provides a relatively constant level of modulating agent release. The amount of modulating agent contained within a sustained release formulation depends upon the site of implantation, the rate and expected duration of release and the nature of the condition to be treated or prevented.

Pharmaceutical compositions of the present invention may be administered in a manner appropriate to the disease to be treated (or prevented). Appropriate dosages and a suitable duration and frequency of administration will be determined by such factors as the condition of the patient, the type and severity of the patient's disease and the method of administration. In general, an appropriate dosage and treatment regimen provides the modulating agent(s) in an amount sufficient to provide therapeutic and/or prophylactic benefit. Within particularly preferred embodiments of the invention, a modulating agent or pharmaceutical composition as described herein may be administered at a dosage ranging from 0.001 ng to 50 mg/kg body weight. For topical administration, a cream typically comprises an amount of modulating agent ranging from 0.00001% to 1%. Fluid compositions may contain about 0.01 ng/ml to 5 mg/ml of modulating agent. Appropriate dosages may generally be determined using experimental models and/or clinical trials. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

As noted above, contact of a cell with a modulating agent as described herein is intended to inhibit or prevent disease states in which dysregulation of AP-1 mediated transactivation is implicated. In particular, inflammatory and immune responses will be treatable using the present invention as AP-1 mediated transactivation is integral to these physiological processes. In addition, AP-1 dysregulation is known to play a role in promoting cancerous diseases and modulating agents modulating, preferably inhibiting, AP-1 mediated transactivation will be effective in retarding the progress of cancerous conditions and, quite possibly, preventing cancerous diseases (as a chemopreventive). Within such methods, a modulating agent may be administered to a patient that is at risk for developing a particular disease (but without detectable symptoms), or may be administered following diagnosis of the disease, based on clinical parameters that are accepted by those skilled in the art. In general, a modulating agent is administered in an amount sufficient to delay the onset, slow the progression or effect all improvement in symptoms of the disease.

The utility of the above-described agents and methods of treating a disease state are further substantiated by the recently described report demonstrating a correlation between Notch-1 activity and AP-1 activity in carcinoma cells. See Talora et al., (2002) Genes & Development 16:2252-2263. It has been shown that the expression of endogenous Notch-1 is markedly reduced in a panel of cervical carcinoma cells and Notch-1 expression is reduced or absent in invasive cervical cancers. Conversely, expression of activated Notch-1 causes strong growth inhibition of human papillomaviruses (HPV)-positive, but not HPV-negative, cervical carcinoma cells, but exerts no such effects on other epithelial tumor cells. It was further observed that increased Notch-1 signaling, but not Notch-2, causes a dramatic down-modulation of HPV-driven transcription of the E6/E7 viral genes, through suppression of AP-1 activity. Thus, Notch-1 has been observed to exert specific protection against HPV-induced transformation in an AP-1 dependent manner and down-regulation of Notch-1 expression is likely to play an important role in late stages of HPV-induced carcinogenesis.

The following Examples are offered by way of illustration and not by way of limitation.

### EXAMPLES

A. Materials and Methods

Plasmids—The pBabe-NIC-1 (pNIC-1) expression vector encoding constitutively active Notch-1 (NIC-1) was described previously (36,37,51). This vector was derived from the pBabe-puro retroviral vector and includes a cDNA sequence encoding amino acids 1759-2556 of human Notch-1 with a Myc tag fused to its carboxyl terminus. Human NIC-1 deletion mutants were generated by PCR using a full-length human Notch-1 expression vector as the template. The Notch-dependent reporter plasmid containing four CBF1 binding sites and an simian virus 40 promoter fused to luciferase (p4×CBF1luc) was described previously (52). The AP-1 reporter plasmid (p1×AP1luc) containing a collagenase promoter fragment (−73/+67) with a single AP-1 binding site in the luciferase reporter vector pGL2-basic (Promega) was described previously (53). The NF-κB reporter plasmid (p3×κBluc) containing three NF-κB binding sites and a minimal promoter fused to luciferase was a kind gift of Dr. Shigeki Miyamoto (University of Wisconsin Medical School). The pBabe-H-Ras(12V) expression vector encoding constitutively active H-Ras was a kind gift of Dr. Charming Der (University of North Carolina-Chapel Hill). This vector was derived from the pBabe-puro retroviral vector and includes a cDNA encoding H-Ras with a Gly to Val mutation at amino acid 12.

Cell Culture—The human erythroleukemia cell line K562 was propagated in Iscove's modified Eagle's medium (Biofluids) containing 10% fetal bovine serum and 1% penicillin/streptomycin (Life Technologies, Inc) (complete IMEM). HeLa cells were maintained in Dulbecco's modified Eagle's medium (Biofluids) containing 10% fetal bovine serum and 1% penicillin/streptomycin (Life Technologies, Inc) (complete DMEM). Cells were grown in a humidified incubator at 37° C., in the presence of 5% carbon dioxide.

Indirect Immunofluoresence—HeLa cells (2.5×10⁶) were seeded into six-well plates and were transfected with 2 μg of the indicated NIC-1 construct. Cells were transfected using 8 μl Lipofectamine in a total volume of 2 ml of Optimem; 24 h post-transfection, cells were plated at 5.0×10⁴ cells per well on four-chamber glass slides. IF was performed as described (37) using bTAN15A (54) for the primary antibody, followed by incubation with a donkey-anti-rat Cy3-conjugated secondary antibody. Proteins were photographed on a Zeiss Axiophot fluorescent microscope with a Hamamatsu digital camera at 400× magnification.

Stable Transfection—K562 cells were stably transfected by electroporation with a Bio-Rad Gene pulser electroporator. Cells (5×10⁶) were washed with ice-cold PBS, resuspended in 0.5 ml of ice-cold PBS, mixed with 5 μg linearized plasmid DNA, and subjected to electroporation (960 microfarad; 220 V) in a 0.4 cm-wide electroporation cuvette (BTX). pBabe and pNIC-1 were linearized with NotI. Cells were then added to 20 ml of complete IMEM, grown for 48 h, and diluted in complete IMEM containing 1.5 μg/ml puromycin (pools of K562-Babe and K562-NIC-1 cells). Stably transfected cells were analyzed for erythroid differentiation as soon as the pools were generated by benzidine staining to reduce the probability of phenotypic changes that may result from prolonged growth.

Retroviral Infection—Modified 293 human embryonic kidney cells were grown in 10 cm dishes until they were subconfluent and were cotransfected with plasmid DNA (15 μg) and pMD.G (6 μg) by the calcium phosphate transfection method as described previously (55). The medium was changed once after 10 h of transfection to remove the calcium phosphate. The pMD.G expression vector encodes the viral envelope protein VSV-G. The modified 293 cells were previously stably transfected with pol and gag genes (gift of Shigeki Miyamoto, University of Wisconsin Medical School). After additional incubation for 12 h, the medium was removed and K562 cells (10 ml, 3×10⁵/ml) were added with polybrene (4 μg/ml) in complete IMEM and incubated for 36 h. The infected cells were separated from adherent 293 cells and then subjected to immunoprecipitation analysis.

Transient Transfections—K562 cells (5×10⁵) were collected by centrifugation at 240×g for 8 min at 4° C. and resuspended in 4 ml of complete IMEM. Plasmid DNAs (1 μg of reporter and 2 μg of effector) were added to 150 μl of IMEM, incubated with Superfect (4 μl/1 μg DNA; Qiagen) for 10 min at room temperature and then added to cells.

For transient transfection of HeLa cells, cells (2×10⁵) were seeded in a 6-well plate one day prior to transfection. On the day of transfection, medium was removed, cells were washed once with ice-cold PBS, and 600 μl of complete DMEM was added. Plasmid DNAs (1 μg of reporter and 2 μg of effector) were added to 150 μl DMEM, incubated with 12 μl Superfect (Qiagen) for 10 min at room temperature and then added to cells. After incubating for 3 h, the mixture was removed, cells were washed once with ice-cold PBS, and 4 ml of fresh complete DMEM was added.

For each transfection, cells were incubated for 26 h after transfection and then treated with TPA (final concentration: 5 nM) or the vehicle (DMSO). After incubating for another 12 or 16 h, cells were harvested and assayed for luciferase activity. Luciferase activity was normalized by the protein content of the lysates, determined by Bradford assay using γ-globulin as a standard.

Northern Blotting—Total RNA from K562-Babe and K562-NIC-1 cells was extracted with Triazol (Life Technologies, Inc.). Ten μg of RNA per sample was electrophoresed on a 1% agarose, 6.6% formaldehyde gel and then transferred overnight to a Magnacharge nylon membrane (Osmonics). RNA was cross-linked to the membrane by UV irradiation. Membranes were prehybridized for 30 min at 60° C. in ExpressHyb hybridization solution (Clontech). Hybridization was performed using high-specific activity ³²P-labeled probes generated by random priming cDNA fragments. Blots were washed three times in 2×SSC/1% SDS, followed by three times in 0.2×SSC/0.1% SDS (30 min per wash). Radioactivity was quantitated by PhosphorImager analysis with ImageQuant software (Molecular Dynamics).

Western Blotting—To detect the expression of Myc-tagged wild type NIC-1 and NIC-1 mutants, whole cell lysates were prepared in Nonidet P-40 lysis buffer (50 mM Hepes, pH 7.4, 1 mM EDTA, 150 mM NaCl, 10% glycerol, 1% Nonidet P-40, 2 mM DTT, 0.2 mM phenylmethanesulfonyl fluoride, and 20 μg/ml leupeptin). Lysates were cleared by centrifugation at 13,000×g for 30 min at 4° C. Supernatants were split into two aliquots and immunoprecipitated with either preimmune serum or anti-NIC 925 polyclonal antibody. Anti-NIC is a rabbit polyclonal antiserum directed against amino acids 1759-2095 of human Notch-i (37). Immune complexes were collected by adsorption to protein A-Sepharose. Proteins were resolved by SDS-PAGE on an 8% acrylamide gel. The proteins were transferred to an Immobilon P membrane (Millipore) and detected by immunoblotting with the anti-Myc tag monoclonal antibody 9E10. CBF1 was detected by immunoblotting with anti-CBF1 polyclonal antisera (Lam & Bresnick, unpublished data).

To measure the phosphorylation state of components of the MAPK pathway, K562-Babe and K562-NIC-1 cells (1×10⁶) were collected after treatment with 5 nM TPA or DMSO for 30 min. Cells were washed once with ice-cold PBS, cell pellets were resuspended in 30 μl ice-cold PBS and were immediately boiled in 70 μl SDS sample buffer for 10 min. Proteins (10 μl) were resolved by SDS-PAGE on a 10% acrylamide gel and transferred to Immobilon P membrane (Millipore). After blocking membranes in 5% nonfat dry milk in TBST (10 mM Tris pH 8.0, 150 mM NaCl, 0.3% Tween-20), membranes were incubated with primary antibody (diluted 1:1000 in dry milk/TBST). The following antibodies were used: ERK1/2 and phospho-specific antibodies for ERK1/2, p38, c-Jun (Ser73) (New England BioLabs—product numbers 9102, 9101, 9211, and 9260, respectively); p38, JNK1, c-Jun, c-Fos, and phospho-specific antibody for JNK (Santa Cruz Biotechnology—product numbers sc-535, sc-474, sc-45, sc-253, and sc-6254, respectively). Protein A-horseradish peroxidase conjugate (BioRad) was added at a dilution of 1:2500 in 5% dry milk/TBST to membranes incubated with anti-ERK1/2 and anti-phosphoERK1/2. Horseradish peroxidase-conjugated donkey anti-goat IgG (Santa Cruz Biotechnology) was added at 1:3500 to membranes incubated with anti-JNK1. Horseradish peroxidase-conjugated goat anti-mouse IgG (Santa Cruz Biotechnology) was used at 1:5000 for membranes incubated with anti-phospho-JNK. Horseradish peroxidase-conjugated goat anti-rabbit IgG (Santa Cruz Biotechnology) was used at 1:5000 for membranes incubated with other antibodies. Antigen-antibody complexes were detected with ECL Plus™ (Amersham Life Science) according to manufacturer's instructions.

Preparation of Nuclear Extracts—Nuclear extracts were prepared as described previously (56). K562-Babe and K562-NIC-1 cells were harvested by centrifugation for 10 min at 150×g. Cells were washed once with ice-cold PBS and resuspended in 1.5 volumes of nuclei lysis buffer (10 mM Tris-HCl, pH 7.5, 10 mM NaCl, 3 mM MgCl₂, and 0.2% Nonidet P-40) on ice for 3 min. Nuclei were collected by centrifugation for 5 min at 600×g. Nuclei were washed by gentle resuspension in 1.5 volumes of nuclei wash buffer (10 mM Tris-HCl, pH 7.5, 10 mM NaCl, and 3 mM MgCl₂) and then collected by centrifugation for 4 min at 600×g. Nuclei were immediately resuspended in an equal volume of low KCl extract buffer (20 mM HEPES, pH 7.5, 20 mM KCl, 1.5 mM MgCl₂, 0.2 mM EDTA, and 25% glycerol), and 1.33 volumes of the same buffer containing 1.2 M KCl was added dropwise. Nuclei were extracted for 45 min at 4° C. with constant mixing. The suspension was then centrifuged for 30 min at 150,000×g. Aliquots of the supernatant were frozen on dry ice and stored at −80° C. The protein concentration as measured by the Bradford assay, with y-globulin as a standard ranged from 4 to 10 mg/ml. DTT (5 mM), phenylmethanesulfonyl fluoride (0.5 mM), leupeptin (20 μg/ml), β-glycerophosphate (800 μM), sodium vanadate (1 mM), and sodium molybdate (50 μM) were included in all buffers.

Electrophoretic Mobility Shift Assay—EMSA assays were done as described previously (54). AP-1 DNA binding activity was measured by EMSA with a double-stranded end-labeled oligonucleotide (ACCTGTGCTGAGTCACTGGAG) containing a high affinity AP-1 binding site. The specificity of DNA binding was assessed by competition with a 100-fold excess of the AP-1 oligonucleotide or an oligonucleotide (HBP) (TTTAGTCAGGTGGTCAGCTTCT) containing a high-affinity USF binding site (57). To assess the composition of the AP-1 complex, extracts were preincubated with 4 μg of anti-c-Jun or anti-c-Fos antibodies, or purified rabbit IgG for 2 h at 4° C. Radiolabeled AP-1 oligonucleotide was then added, and samples were incubated for 20 min at room temperature. Samples were resolved on a 6.3% nondenaturing polyacrylamide gel in 0.75×TAE buffer at 4° C. DNA binding activity was quantitated by PhosphorImager analysis with ImageQuant software (Molecular Dynamics).

B. Repression of Endogenous AP-1 by the Notch-1 Intracellular Domain—The present inventors previously showed that NIC-1 represses transcriptional activation of IL-8 upon erythroid maturation of K562 erythroleukemia cells (51). These cells express endogenous Notch-1 and are competent to carry out strong CSL-dependent transcriptional activation (51). To investigate mechanisms underlying the repression, it was tested whether NIC-1 antagonizes factors required for induction of IL-8 transcription. AP-1 (58) and NF-κB binding sites (59) on the IL-8 promoter are critical for transcriptional activation of IL-8 in response to diverse signals, although the relative importance of the two sites differs in different cell systems (60,61).

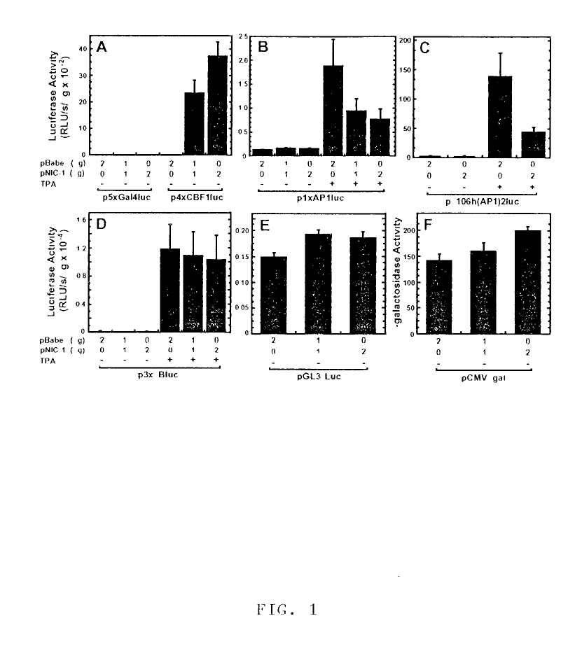

AP-1- and NF-κB-responsive luciferase reporter constructs and a NIC-1 expression vector were transiently cotransfected into K562 cells. Previously, it was shown that that this NIC-1 expression vector confers low level expression of NIC-1 protein in K562 cells (51) and other cell types (36,37). NIC-1 expression in K562 cells strongly activated transcription of a luciferase reporter that binds endogenous CSL proteins (FIG. 1A). Treatment of cells with the phorbol ester TPA to activate endogenous AP-1 strongly induced the activity of an AP-1 reporter containing a collagenase 1 (MMP1) promoter with a single AP-1 site (62). Under identical conditions in which the CSL reporter was activated by NIC-1, NIC-1 repressed AP-1 reporter activity (FIG. 1B). It was asked whether repression was dependent upon the context of the AP-1 binding site within the promoter of the reporter. The degree of repression seen with a distinct AP-1 reporter, containing tandem AP-1 binding sites upstream of a β-globin promoter (63) (FIG. 1C), was comparable to that seen with the MMP1 promoter (FIG. 1B), suggesting that repression is not context-dependent. Although the repression was strong with both AP-1 reporters, a component of the TPA-induced AP-1 reporter activity (˜30%) was insensitive to NIC-1.

As IL-8 transcription is also controlled via an NF-κB binding site on the IL-8 promoter (58,59), the inventors asked whether NIC-1 affects NF-κB-dependent transcription. Previous studies in different systems showed that NIC-1 can repress (29,30) and activate NF-KB (31). Upon transient transfection into K562 cells, an NF-KB reporter gene containing three NF-KB binding sites was strongly activated by treatment of cells with TPA (FIG. 1D). In contrast to the AP-1 reporters, NIC-1 had no effect on NF-κB-dependent reporter activity (FIG. 1D). To further assess the specificity of the NIC-1-mediated AP-1 repression, it was asked whether NIC-1 influenced the activity of a constitutively active promoter, the human Aγ globin promoter (pGL3γLuc) (FIG. 1E) and a constitutively active enhancer, the CMV enhancer (pCMVβgal) (FIG. 1F). TPA treatment increased the activity of pGL3γLuc by ˜3 fold and strongly increased the activity of pCMVβgal. NIC-1 increased the basal activity of pGL3γLuc by ˜80% without affecting the TPA-induced activity. NIC-1 had no effect on the basal activity of pCMVβgal but increased the TPA induced activity by ˜2 fold. Thus, NIC-1 represses AP-1-dependent transactivation in a context-independent manner in transient transfection assays. The lack of repression of NF-κB-dependent transcription and pGL3γLuc and pCMVβgal suggests that there is a considerable degree of specificity for the repression. These results are inconsistent with models in which NIC-1 has a general repressive effect on components of the basal transcription machinery or on all forms of activated transcription.

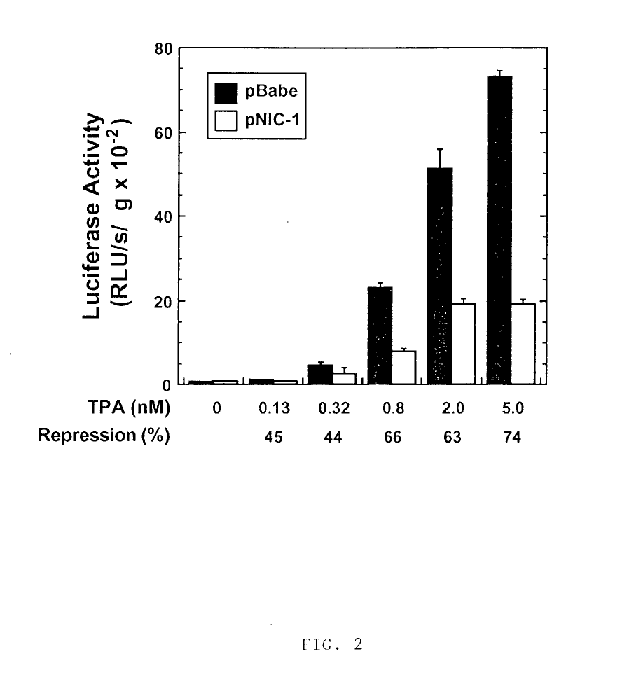

The failure of NIC-1 to completely repress AP-1-mediated transactivation could be due to an intrinsically resistant component of AP-1 activity or the inability of NIC-1 to overcome the strong stimulation of AP-1 activity achieved with a maximally effective TPA concentration. To distinguish between these possibilities, K562 cells were treated with a range of TPA concentrations, and the degree of inhibition by NIC-1 was compared under conditions of submaximal and maximal stimulation (FIG. 2). At all TPA concentrations, a resistant component of activity was apparent, and the degree of inhibition was not higher upon submaximal stimulation of AP-1. These results show that NIC-1 inhibits 70% of the TPA-inducible AP-1 activity, while a second component of the AP-1 activity is resistant to repression by NIC-1.

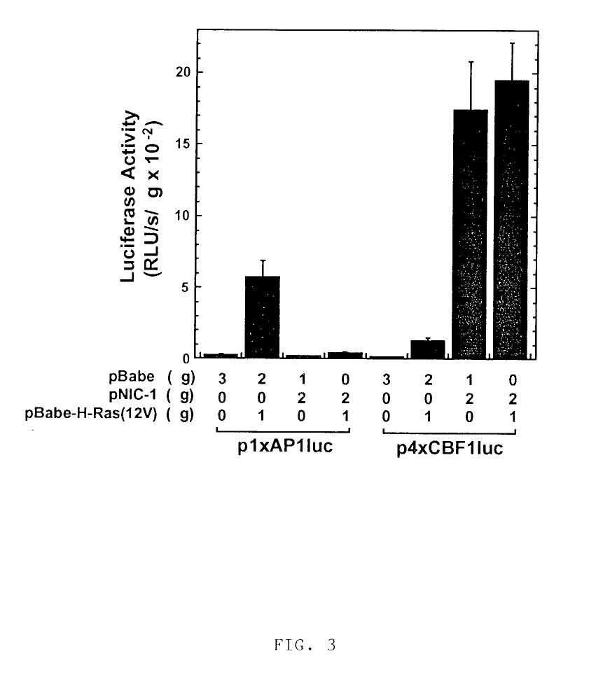

To determine whether AP-1 activity induced by a distinct stimulus was inhibited by NIC-1 and whether a component of the activity was resistant to NIC-1, the inventors activated endogenous AP-1 by transient expression of constitutively active H-Ras(12V). H-Ras(12V) activated AP-1 reporter activity, and NIC-1 almost completely inhibited H-Ras(12V)-activated AP-1 (FIG. 3). H-Ras(12V) expression slightly activated CSL-dependent reporter activity, in the absence of exogenous NIC-1, and did not significantly influence NIC-1-dependent activation of the CSL reporter. Thus, activation of AP-1 by TPA or H-Ras(12V) was strongly inhibited by NIC-1. However, the NIC-1-resistant component of AP-1 activity (FIGS. 1 and 2) was dependent on the mode of AP-1 activation, being unique to activation by TPA.

C. Is Inhibition of AP-1-mediated Transactivation by NIC-1 Physiologically Relevant?

As described in the Introduction, several reports have provided evidence for functional crosstalk between Notch and Ras pathways, establishing a strong precedent for physiological Notch-Ras interactions. Activation of JNK and p38, downstream of Ras, leads to phosphorylation of serines 63 and 73 on the amino-terminus of c-Jun (and conserved sites of other Jun family members), thereby stimulating AP-1-mediated transcription. The inventors' discovery that NIC-1 represses AP-1-mediated transcription may reflect a previously unrecognized component of Notch-Ras crosstalk. It was reasoned that if the repression of AP-1-mediated transactivation by NIC-1 is physiological, repression would not be unique to K562 cells, endogenous AP-1 target genes would be repressed, and repression would not require higher concentrations of NIC-1 than for activation of CSL-dependent transcription. These issues were addressed in the following experiments.

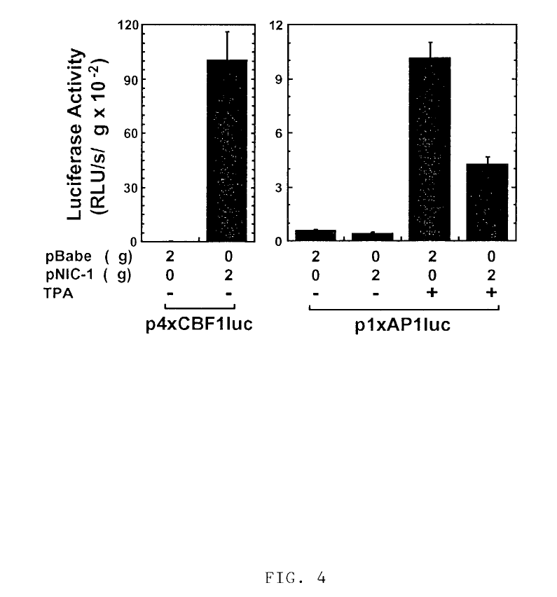

To assess whether repression of AP-1-mediated transactivation by NIC-1 was unique to K562 cells, the inventors asked whether NIC-1 represses endogenous AP-1 in HeLa cells (FIG. 4). NIC-1 strongly activated CSL-dependent reporter activity in HeLa cells. AP-1 reporter activity was strongly induced upon treatment of HeLa cells with TPA. Similar to K562 cells (FIGS. 1 and 2), NIC-1 repressed AP-1-mediated activation, with a component of the activity being resistant to NIC-1. Thus, the repression of AP-1-mediated activation by NIC-1 is not unique to K562 cells, suggesting that repression would be apparent in diverse systems. As AP-1 controls the expression of a plethora of genes mediating immune and inflammatory responses, and NIC-1 has important activities to control immune cell function, crosstalk between Notch and AP-1 pathways would likely have important biological consequences.

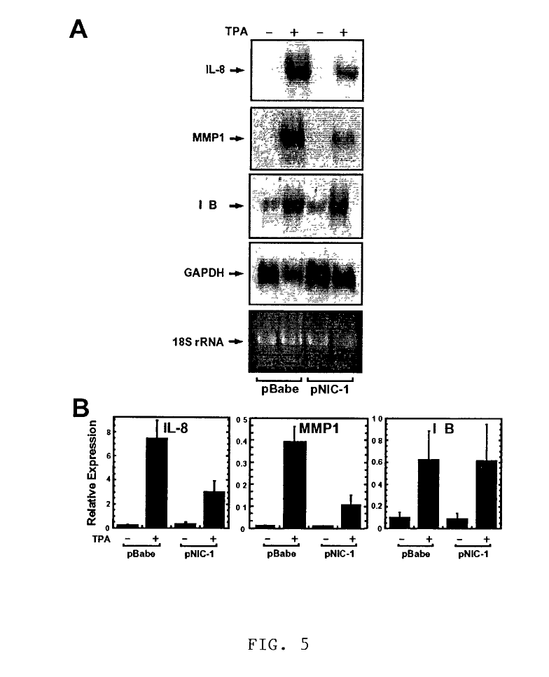

As mentioned above, induction of endogenous IL-8 expression upon erythroid maturation of K562 cells was repressed by stably expressed NIC-1 (51). To define whether NIC-1 deregulates endogenous AP-1 target genes (64,65) in a context that is not confounded by the complexities of cellular differentiation, IL-8 and MMP1 were activated by treatment of K562-Babe and K562-NIC-1 cells with TPA, and steady-state mRNA levels were measured by Northern blotting. Maximal induction of IL-8 by TPA requires both AP-1 and NF-κB binding sites, and the relative importance of the sites varies in different systems (58,59). MMP1 is a prototypical AP-1 target gene, although Ets factors can activate MMP1 via synergism with AP-1 (66), or repress (67) MMP1. TPA treatment strongly induced IL-8 and MMP1 transcript levels in K562-Babe cells containing a stably transfected empty vector, whereas induction was considerably lower in K562-NIC-1 cells containing stably transfected NIC-1 (FIG. 5). The degree of repression of endogenous IL-8 and MMP1 transcription was similar to that of the transient transfection assays of FIGS. 1 and 2. Since the AP-1-responsive pβ106h(AP1)2luc reporter of FIG. 1 and the IL-8 promoter lack Ets sites, NIC-1 does not require coupled AP-1 and Ets sites to confer repression.

To investigate the specificity of the repression in a chromosomal context, the levels of IκBα transcripts after TPA treatment of K562-Babe and K562-NIC-1 cells were measured. IκBα is a prototypical NF-KB target gene (68), and TPA activates IκBα transcription via a mechanism involving NF-κB activation. NIC-1 had no effect on TPA induction of IκBα transcripts, consistent with the experiment of FIG. 1D showing no effect of NIC-1 on TPA induction of an NF-κB reporter in transient transfection assays. The failure of NIC-1 to influence NF-κB-driven transcription in K562 cells suggests that previous reports of NIC-1-mediated repression (29,30) and activation of NF-κB-dependent transcription (31) reflect cell-type specific actions. The inventors' results show that NIC-1 represses the endogenous AP-1 target genes IL-8 and MMP1, and the lack of effect of NIC-1 on induction of IκBα confirms the specificity of the response.

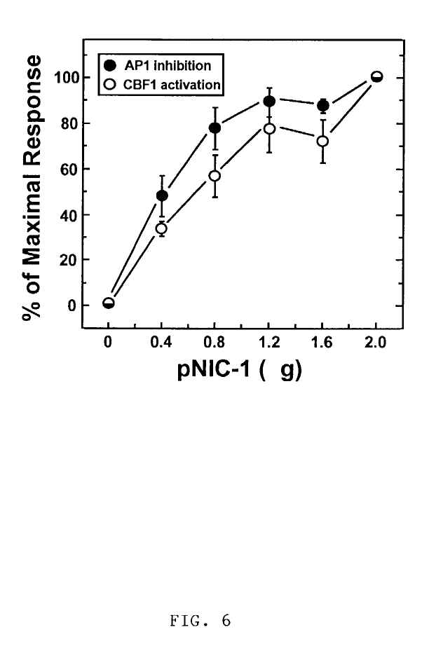

If repression of AP-1-mediated transactivation by NIC-1 is physiologically relevant, repression should occur at NIC-1 concentrations resembling that required to activate CSL-dependent transcription. On the other hand, if repression requires considerably higher concentrations of NIC-1, this would be inconsistent with a physiological mechanism. To address this issue, the inventors compared the concentrations of NIC-1 expression vector required for CSL-dependent activation and AP-1 repression (FIG. 6). Transfection of K562 cells with increasing amounts of NIC-1 expression vector, while maintaining a constant total DNA concentration, induced a concentration-dependent activation of CSL-dependent reporter activity. Similarly, increasing amounts of NIC-1 expression vector decreased AP-1-dependent reporter activity as a function of vector concentration. The concentration response curves for CSL-dependent activation and AP-1 repression were similar. However, the curve for AP-1 repression was slightly shifted to the left, showing that slightly less NIC-1 expression vector was required to achieve a comparable degree of AP-1 repression versus CSL-dependent activation. Thus, at NIC-1 concentrations capable of conferring CSL-dependent activation, the well established physiological action of NIC-1, NIC-1 represses AP-1, providing strong evidence that AP-1 repression would occur under physiological conditions. Taken together with the facts that repression occurs in multiple cell types and endogenous AP-1 target genes are repressed, it is likely that NIC-1 engages in physiological crosstalk with the AP-1 pathway. It is therefor of intrinsic interest to elucidate molecular mechanisms underlying the crosstalk.

D. Requirements for Repression of AP-1-Mediated Transactivation by NIC-1—

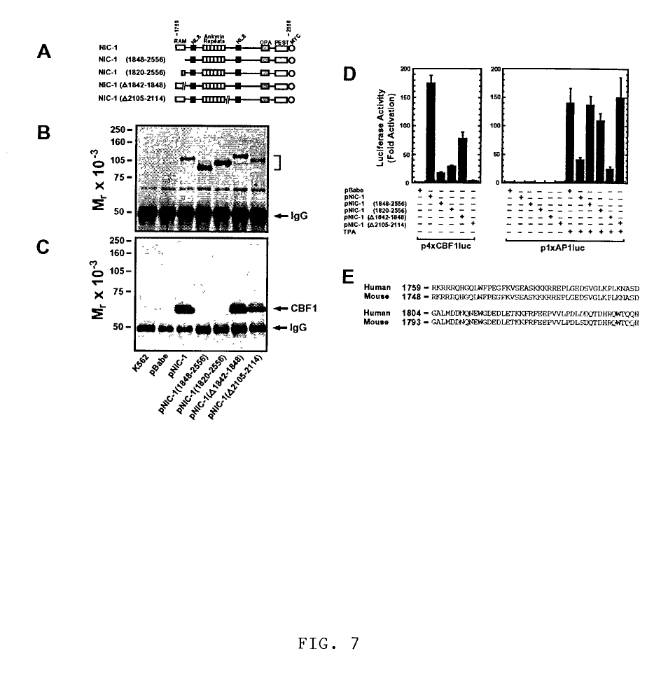

NIC-1 has multiple conserved domains that could potentially mediate AP-1 repression. The sole function ascribed to the RAM domain is high-affinity CSL binding (69,70), which accordingly imparts a requirement for the RAM domain in CSL-dependent activation. To define amino acids of NIC-1 required for repression, NIC-1 mutants were generated lacking the RAM domain [NIC-1(1848-2556)], containing only a 29 amino acid segment of the RAM domain [NIC-1(1820-2556)], containing a seven amino acid deletion within the RAM domain [NIC-1(Δ1842-1848)], and containing a ten amino acid deletion downstream of the ankyrin repeats [NIC-1(Δ2105-2114)] (FIG. 7A). The expression of wild-type NIC-1 and NIC-1 mutants was assessed by immunoprecipitation with an anti-NIC-1 antibody with extracts isolated from transfected K562 cells, and immunoprecipitated proteins were detected by Western blotting with an anti-myc antibody. All mutants were expressed, and the expression levels did not differ greatly (FIG. 7B). The blot was also probed with anti-CBF1 antisera to assess the recovery of CBF1 in the immunoprecipitates (FIG. 7C). CBF1 coimmunoprecipitated with wild-type NIC-1, NIC-1(Δ1842-1848), and NIC-1(Δ2105-2114). In contrast, almost no CBF1 was recovered upon immunoprecipitation of NIC-1(1848-2556) and NIC-1(1820-2556), which lack the entire RAM domain and a major portion of the RAM domain, respectively.

The mutants were compared to wild-type NIC-1 for their ability to activate CSL-dependent transcription and to repress AP-1. As expected, NIC-1(1848-2556) only weakly induced CSL reporter activity. Surprisingly, NIC-1(1848-2556) only weakly repressed AP-1 reporter activity (FIG. 7D). NIC-1(1820-2556) had a similar behavior, being strongly impaired in both CSL-dependent activation and AP-1 repression. Thus, analysis of constructs with complete and partial RAM domain deletions revealed a critical requirement of RAM domain sequences for CSL-dependent activation and AP-1 repression. NIC-1(Δ1842-1848) conferred less CSL-dependent activation than wild-type NIC-1, whereas it repressed AP-1 slightly better than wild-type NIC-1. Intriguingly, amino acids 1842-1848 are selectively required for maximal CSL-dependent activation but not for repression. An additional mutant, NIC-1(Δ2105-2114), known to be strongly impaired in conferring transactivation (37) was also tested. As expected NIC-1(Δ2105-2114) weakly activated CSL-dependent reporter activity, similar to NIC-1 (1848-2556) and NIC-1(1820-2556); NIC-1(Δ2105-2114) did not repress AP-1 reporter activity. Since CBF1 coimmunoprecipitated with NIC-1(Δ2105-2114), and NIC-1(Δ2105-2114) was not competent for AP-1 repression, clearly CBF1 binding is insufficient for AP-1 repression. These results provide evidence that sequences within the highly conserved RAM domain of NIC-1 and amino acids 2105-2114 are critical for CSL-dependent activation and AP-1 repression. Despite these common sequence requirements for CSL-dependent activation and AP-1 repression, the behavior of NIC-1 (Δ1842-1848) is consistent with distinct, but overlapping, sequence requirements within the RAM domain. The RAM domain requirement for AP-1 repression constitutes a previously undescribed activity of this evolutionarily conserved domain (FIG. 7E); the RAM domain was only known to mediate CSL binding and CSL-dependent activation.

As noted above, NIC has been shown to inhibit H-Ras-mediated activation of E47-dependent transactivation in transient transfection assays (21). In that study, it was also shown in transient assays in 3T3 cells that NIC-2 inhibited transactivation mediated by the GAL4 DNA binding domain fused to a portion of c-Jun, and that inhibition did not require the RAM domain. This contrasts with our results in which the intact RAM domain (amino acids 1759-1847) and a portion of the RAM domain (amino acids 1759-1819) were absolutely required for repression of endogenous AP-1. This difference may reflect cell-type specific differences in the behavior of NIC, different influences of NIC on GAL4-c-Jun and endogenous AP-1, or differences between activities of NIC-1 and NIC-2. The inventors assessed the impact of NIC-1 on transactivation mediated by GAL4 fused to the c-Jun activation domain (GAL4-c-Jun) in transient assays in K562 cells. NIC-1 did not significantly inhibit GAL4-c-Jun-mediated transactivation (data not presented).

Given that NIC-1 localizes predominantly to the nucleus, the inventors reasoned that repression might occur within the nucleus. However, AP-1 is known to be activated via phosphorylation of amino terminal serines of c-Jun and Jun family members, and therefore it is conceivable that NIC-1 disrupts membrane or cytoplasmic signaling events required for AP-1 phosphorylation and subsequent activation. Importantly, the experiments of FIGS. 1 and 5 used TPA to activate NF-κB-dependent transcription, and NIC-1 had no effect on the TPA-dependent induction. This suggests that if NIC-1 inhibits TPA-dependent signaling events, potentially, these events would not be shared by the NF-κB and AP-1 activation pathways.

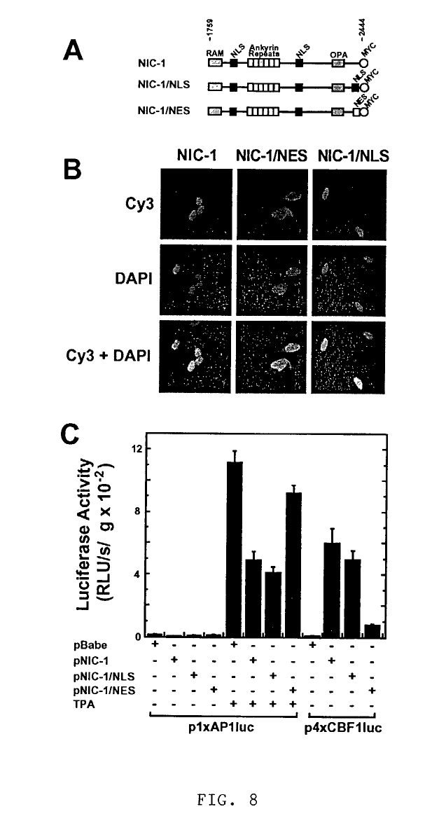

To define whether repression requires nuclear localization of NIC-1, NIC-1 derivatives were tested in which NES or NLS sequences were engineered at the carboxyl terminus (FIG. 8A). It was shown previously by indirect immunofluorescence assays that NIC-1I/NLS resembles NIC-1 in having a predominant nuclear localization, whereas NIC-1/NES localizes to the cytoplasm and to the nucleus (37). Given the established function of NES sequences (71), it is likely that the cytoplasmic and nuclear distribution of NIC-1/NES reflects active shuttling of NIC-1/NES between the two cell compartments. We examined the subcellular localization of these NIC-1 derivatives in HeLa cells and tested their ability to activate CSL-dependent transcription and to repress AP-1. The subcellular localization of the constructs (FIG. 8B) was similar to that described previously (37). While NIC-1/NLS resembled NIC-1 in activating CSL-dependent reporter activity and repressing AP-1 reporter activity, NIC-1/NES only weakly activated CSL-dependent reporter activity and weakly repressed AP-1 reporter activity (FIG. 8C). These results provide a correlation between predominant nuclear localization and strong repression of AP-1, supporting a model in which repression occurs within the nucleus.

E. Does NIC-1 Inhibit Signaling Events Required for AP-1 Activation?

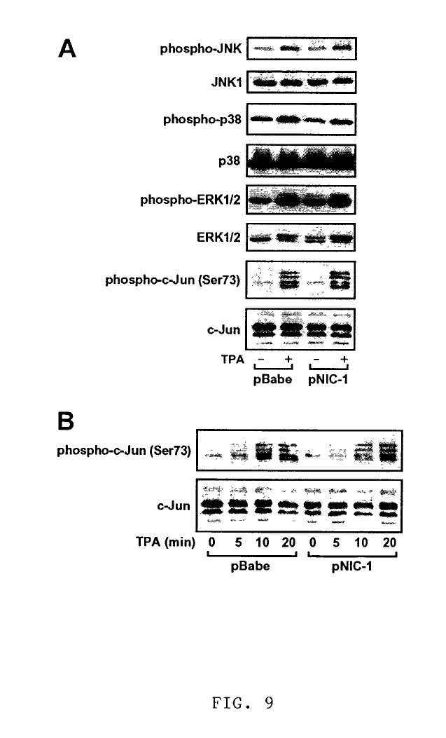

If repression of AP-1 by NIC-1 occurs within the nucleus, this would be inconsistent with an inhibitory effect of NIC-1 on membrane and cytoplasmic signaling events necessary for AP-1 activation. To define the influence of NIC-1 on such signaling events, the inventors measured the phosphorylation state of relevant signaling components by Western blot analysis with phospho-specific antibodies. An inhibitory effect of NIC-1 on signaling would be manifested by disrupted signaling downstream of the inhibited step and normal signaling upstream of the inhibited step. Multiple MAPKs have been reported to be activated by TPA including JNK, p38, and ERK1/2. Analysis of the phosphorylation state of these MAPK subtypes, under identical growth conditions as the transient transfection and Northern analyses, revealed that TPA induced phosphorylation of these components to varying degrees, but had no measurable effect on the expression levels of the components (FIG. 9A). Stably transfected NIC-1 did not affect TPA-induced or basal phosphorylation of JNK or p38; basal and TPA-induced ERK1/2 phosphorylation were slightly higher in K562-NIC-1 versus K562-Babe cells. As the identical stably transfected cells that were subjected to Western blot analysis were analyzed by Northern blotting for induction of IL-8 and MMP1 expression, and these genes were repressed by NIC-1 (FIG. 5), it is unlikely that impaired phosphorylation of MAPKs is the mechanism underlying AP-1 repression.

Activation of MAPKs can result in nuclear translocation of the activated enzymes (72). As noted above, one consequence of JNK and p38 activation is phosphorylation of serines 63 and 73 of c-Jun and conserved serines of Jun family members. The inventors tested whether Jun phosphorylation was impaired in K562-NIC-1 cells using antibodies specific for phosphorylated serine 73 of c-Jun and the corresponding site of JunD and phosphorylated serine 63 of c-Jun. NIC-1 did not affect c-Jun protein levels nor did it influence phosphorylation at either site (serine 73, FIG. 9A; serine 63, data not shown). Thus, it is unlikely that altered synthesis or disrupted phosphorylation of Jun proteins causes decreased AP-1 activity. The lack of effect of NIC-1 on serine 63 and 73 phosphorylation is consistent with the failure of NIC-1 to inhibit JNK and p38 phosphorylation; inhibition of JNK and p38 phosphorylation should decrease phosphorylation of serines 63 and 73 of c-Jun and the corresponding sites of JunD. Furthermore, if NIC-1 inhibited JNK catalytic activity, this would also be expected to decrease serine 63 and 73 phosphorylation. NIC-1 also did not affect Fos protein levels (FIG. 9A), inconsistent with a mechanism in which NIC-1 decreases AP-1 activity by reducing Fos expression.

One caveat of the Western blot experiments of FIG. 9A is that NIC-1 could potentially modulate temporal aspects of phosphorylation, and this might not be evident from steady-state measurements. The inventors therefore examined the time course for phosphorylation of c-Jun (serine 73) and JunD upon TPA treatment of K562-Babe and K562-NIC-1 cells (FIG. 9B). NIC-1 had no effect on the time-dependent induction of phosphorylation, inconsistent with a role for NIC-1 in repressing AP-1 via disruption of signaling events necessary for activation of c-Jun and Jun family members. The failure of NIC-1 to inhibit c-Jun and JunD phosphorylation is consistent with the results of FIG. 8 showing that AP-1 repression requires nuclear localization of NIC-1. Since MAPK activation occurs in the cytoplasm, presumably NIC-1/NES, which localizes in part to the cytoplasm, would be competent to repress AP-1 if disrupted MAPK activation was involved.

F. NIC-1 Does Not Inhibit AP-1 DNA Binding In Vitro

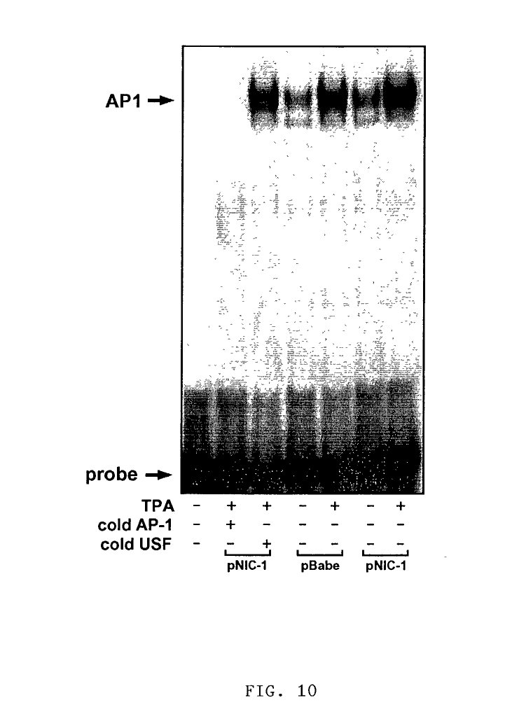

In addition to the phosphorylation of serines 63 and 73 of c-Jun, which is required for transactivation, phosphorylation of c-Jun near the DNA binding domain has been reported to inhibit DNA binding (43). Dephosphorylation would be required to confer high-affinity DNA binding. It was important to test whether this mode of regulation is relevant to the NIC-1-mediated repression of AP-1, since NIC-1 could potentially antagonize dephosphorylation or potentiate phosphorylation, thereby inhibiting DNA binding and transactivation. K562-Babe and K562-NIC-1 cells were treated with TPA to activate AP-1, and nuclear extracts were isolated to measure AP-1 DNA binding activity by EMSA. AP-1 DNA binding activity was strongly induced upon treatment of the cells with TPA, and there were no apparent qualitative or quantitative differences in the AP-1 complexes formed with extracts from K562-Babe and K562-NIC-1 cells (FIG. 10). Both anti-c-Jun and anti-c-Fos antibodies reduced the levels of complex formed, strongly arguing that the complex contains c-Jun and c-Fos subunits. To ensure that AP-1 components were not dephosphorylated upon nuclear extract isolation, phosphatase inhibitors were included in buffers, and this did not influence the AP-1 complexes, nor did it reveal an influence of NIC-1 on DNA binding. Thus, AP-1 complexes from K562-NIC-1 cells have an apparently normal DNA binding activity in vitro, suggesting that impaired AP-1-dependent transactivation is not caused by defective DNA binding. Furthermore, this result is inconsistent with an effect of NIC-1 on the levels of Jun or Fos family members, since reduced levels of these AP-1 components should be evident by reduced AP-1-DNA complex formation.

G. Physiological and Mechanistic Considerations of Notch-AP-1 Crosstalk

AP-1 is essential for transcriptional activation of genes encoding numerous cytokines and enzymes mediating extracellular matrix remodeling, thereby establishing a critical role for AP-1 in immune and inflammatory responses (33,34). A role for Notch signaling in immunity and vascular remodeling has emerged from recent genetic, molecular, and biochemical analysis (27,73-75). As the negative crosstalk between NIC-1 and AP-1 was evident in multiple cell types (FIGS. 1 and 4), endogenous AP-1 target genes were affected (FIG. 5), and similar concentrations of NIC-1 were required for CSL-dependent transcription and AP-1 repression (FIG. 6), it seems reasonable to assume that such crosstalk would occur in diverse physiological contexts. Thus, it is probable that negative crosstalk between Notch and AP-1 pathways would have important implications for immunity, inflammation, vascular remodeling, and potentially other biological processes.

Establishing the physiological implications of the Notch-AP-1 crosstalk may be facilitated by further analysis of the underlying mechanisms. Two models to explain the NIC-1-mediated repression include disruption of AP-1 complex assembly on the chromatin template and impaired coactivator utilization by the AP-1-containing nucleoprotein complex. Given the overlapping sequence determinants for activation and repression, it is possible that the RAM domain interacts with CSL to confer both activities; the only function previously ascribed to sequences within the RAM domain is CSL binding. Alternatively, as amino acids 1842-1848 of the RAM domain are selectively required for activation but not repression, one cannot rule out the possibility that the RAM domain interacts with a unique target to confer repression. The possibility of a distinct target mediating AP-1 repression is reinforced by the observation that NIC-1 (Δ2105-2114) associates with CBF 1 but does not repress AP-1. Thus, CBF 1 binding is not sufficient to confer AP-1 repression.

AP-1 is known to be repressed by steroid hormone signaling pathways (62,76-80). The mechanism of AP-1-steroid receptor crosstalk has required extensive analysis but remains incompletely understood. Nevertheless, it is instructive to compare the influence of steroid receptors and NIC-1 on AP-1. Recently, it was shown that repression of AP-1-mediated transactivation of the collagenase 3 promoter by the ligand-activated glucocorticoid receptor occurs after AP-1 DNA binding (79). The glucocorticoid receptor-interacting coactivator GRIP1 was important for AP-1 repression, and it was proposed that GRIP 1 confers activation and repression of target genes in a context-dependent manner. Based on the failure of NIC-1 to inhibit JNK-dependent phosphorylation of serines 63 and 73 of c-Jun (FIG. 9) and its lack of effect on AP-1 DNA binding in vitro (FIG. 10), the mechanism of AP-1 repression may be analogous to the glucocorticoid receptor scenario, whereby coactivator usage post-DNA binding is an important determinant. AP-1 is known to utilize multiple coactivators including CBP/p300 (76) and Jab1 (47,50). Preliminary experiments show that CBP overexpression does not overcome NIC-1-mediated repression of AP-1, suggesting that NIC-1 does not simply sequester limiting amounts of CBP (data not presented). Jab1 is a component of the COP9 signalsome (81), which has been implicated in multiple regulatory functions including the control of protein degradation. An influence of NIC-1 on COP9 signalsome-dependent AP-1 activation, and more generally on COP9 signalsome function, would have broad implications far beyond the control of AP-1 target genes.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### REFERENCES

1. Kimble, J., and Simpson, P. (1997) Ann. Rev. Cell Dev. Biol. 13, 333-361

2. Artavanis-Tsakonas, S., Rand, M. D., and Lake, R. J. (1999) Science 284, 770-776

3. Mumm, J. S., and Kopan, R. (2000) Dev. Biol. 228, 151-165

4. Artavanis-Tsakonas, S., Delidakis, C., and Fehon, R. G. (1991) Ann. Rev. Cell Biol. 7, 427-452

5. Sestan, N., Artavanis-Tsakonas, S., and Rakic, P. (1999) Science 286, 689-690

6. Milner, L. A., and Bigas, A. (1999) Blood 93, 2431-2448

7. Uyttendaele, H., Ho, J., Rossant, J., and Kitajewski, J. (2001) Proc. Natl. Acad. Sci. USA 98, 5643-5648

8. Kojika, S., and Griffon, J. D. (2001) Exp. Hematology 29, 1041-1052

9. Hsieh, J. D., and Hayward, S. D. (1995) Science 268, 560-563

10. Matsunami, N., Hamaguchi, Y., Yamamoto, Y., Kuze, K., Kangawa, K., Matsuo, H., Kawaichi, M., and Honjo, T. (1989) Nature 342, 934-937

11. Jarriault, S., Brou, C., Logeat, F., Schroeter, E. H., Kopan, R., and Israel, A. (1995) Nature 377, 355-358

12. Nofziger, D., Miyamoto, A., Lyons, K. M., and Weinmaster, G. (1999) Development 126, 1689-1702

13. Zecchini, V., Brennan, K., and Martinez-Arias, A. (1999) Curr. Biol. 9, 460-469

14. Brou, C., Logeat, F., Gupta, N., Bessia, C., LeBail, O., Doedens, J. R., Cumano, A., Roux, P., Black, R. A., and Israel, A. (2000) Mol. Cell 5, 207-216

15. Mumm, J. S., Schroeter, E. H., Saxena, M. T., Griesemer, A., Tian, X., Pan, D. J., Ray, W. J., and Kopan, R. (2000) Mol. Cell 5, 197-206

16. Schroeter, E. H., Kisslinger, J. A., and Kopan, R. (1998) Nature 393, 382-386

17. Petcherski, A. G., and Kimble, J. (2000) Curr. Biol. 10, R471-473

18. Wu, L., Aster, J. C., Blacklow, S. C. , Lake, R., Artavanis-Tsakonas, S., and Griffon, J. D. (2000) Nat. Genet. 26, 484-489

19. Zhou, S., Fujimuro, M., Hsieh, J. J., Chen, L., Miyamoto, A., Weinmaster, G., and Hayward, S. D. (2000) Mol. Cell. Biol. 20, 2400-2410

20. Boehm, T., Foroni, L., Kaneko, Y., Perutz, M. F., and Rabbitts, T. H. (1991) Proc Natl Acad Sci USA 88, 4367-4371

21. Ordentlich, P., Lin, A., Shen, C. P., Blaumueller, C., Matsuno, K., Artavanis-Tsakonas, S., and Kadesch, T. (1998) Mol. Cell. Biol. 18, 2230-2239

22. Fitzgerald, K., Harrington, A., and Leder, P. (2000) Oncogene 19, 4191-4198

23. Berset, T., Hoier, E. F., Battu, G., Canevascini, S., and Hajnal, A. (2001) Science 291, 1055-1058

24. Ross, D. A., and Kadesch, T. (2001) Mol. Cell. Biol. 21, 7537-7544

25. Hing, H. K., Sun, X., and Artavanis-Tsakonas, S. (1994) Mech. Dev. 47, 261-268

26. Uyttendaele, H., Soriano, J. V., Montesano, R., and Kitajewski, J. (1998) Dev. Biol. 196, 204-217

27. Izon, D. J., Punt, J. A., Xu, L., Kamell, F. G., Allman, D., Myung, P. S., Boerth, N. J., Pui, J. C., Koretzky, G. A., and Pear, W. S. (2001) Immunity 14, 253-264

28. Bigas, A., Martin, D. I., and Milner, L. A. (1998) Mol. Cell. Biol. 18, 2324-2333

29. Guan, E., Wang, J., Laborda, J., Norcross, M., Baeuerle, P. A., and Hoffman, T. (1996)J. Exp. Med. 183,2025-2032

30. Wang, J., Shelly, L., Miele, L., Boykins, R., Norcross, M. A., and Guan, E. (2001) J. Immunol. 167, 289-295

31. Cheng, P., Zlobin, A., Volgina, V., Gottipati, S., Osborne, B., Simel, E. J., Miele, L., and Gabrilovich, D. L. (2001) J. Immunol. 167, 4458-4467

32. Bash, J., Zong, W. X., Banga, S., Rivera, A., Ballard, D. W., Ron, Y., and Gelinas, C. (1999) EMBO J. 18, 2803-2811

33. Jochum, W., Passegue, E., and Wagner, E. F. (2001) Oncogene 20, 401-412

34. Shaulian, E., and Karin, M. (2001) Oncogene 20, 2390-2400

35. Young, M. R., Li, J. J., Rincon, M., Flavell, R. A., Sathyanarayana, B. K., Hunziker, R., and Colbum, N. (1999) Proc. Natl. Acad. Sci. USA 96, 9827-9832

36. Capobianco, A. J., Zagouras, P., Blaumueller, C. M., Artavanis-Tsakonas, S., and Bishop, J. M. (1997) Mol. Cell. Biol. 17, 6265-6273

37. Jeffries, S., and Capobianco, A. J. (2000) Mol. Cell. Biol. 20, 3928-3941

38. Bresnick, E. H., Chu, J., Christensen, H. M., Lin, B., and Norton, J. (2000) J. Cell Biochem Suppl, 46-53.

39. Aster, J. C., and Pear, W. S. (2001) Curr. Opin. Hematol. 8, 237-244

40. Smeal, T., Binetruy, B., Mercola, D. A., Birrer, M., and Karin, M. (1991) Nature 354, 494-496

41. Derijard, B., Hibi, M., Wu, I. H., Barrett, T., Su, B., Deng, T., Karin, M., and Davis, R. J. (1994) Cell 76, 1025-1037

42. Carter, A. B., Tephly, L. A., and Hunninghake, G. W. (2001) J. Biol. Chem. 276, 33826-33832

43. Lin, A., Frost, J., Deng, T., Smeal, T., al-Alawi, N., Kikkawa, U., Hunter, T., Brenner, D., and Karin, M. (1992) Cell 70, 777-789

44. Arias, J., Alberts, A. S., Brindle, P., Claret, F. X., Smeal, T., Karin, M., Feramisco, J., and Montminy, M. (1994) Nature 370, 226-229

45. Mayr, B., and Montminy, M. (2001) Nat. Rev. Mol. Cell. Biol. 2, 599-609

46. Vo, N., and Goodman, R. H. (2001) J. Biol. Chem. 276, 13505-13508

47. Claret, F. X., Hibi, M., Dhut, S., Toba, T., and Karin, M. (1996) Nature 383, 453-457

48. Wei, N., and Deng, X. W. (1999) Trends Genet. 15, 98-103

49. Kleeman, R., Hausser, A., Geiger, G., Mischke, R., Burger-Kentischer, A., Flieger, O., Johannes, F. J., Roger, T., Calandra, T., Kapumiotu, A., Grell, M., Finkelmeier, D., Brunner, H., and Bernhagen, J. (2000) Nature 408, 146-147

50. Naumann, M., Bech-Otschir, D., Huang, X., Ferrell, K., and Dubiel, W. (1999) J. Biol. Chem. 274, 35297-35300

51. Lam, L. T., Ronchini, C., Norton, J., Capobianco, A. J., and Bresnick, E. H. (2000) J. Biol. Chem. 275, 19676-19684

52. Hsieh, J. J., Henkel, T., Salmon, P., Robey, E., Peterson, M. G., and Hayward, S. D. (1996) Mol. Cell. Biol. 16, 952-959

53. Li, J. J., Dong, Z., Dawson, M. I., and Colbum, N. (1996) Cancer Res. 56, 483-489

54. Zagouras, P. S., Stifani, C. M., Blaumueller, M. L., Carcangiui, M. L., and Artavanis-Tsakonas, S. (1995) Proc. Natl. Acad. Sci. USA 92, 6414-6418

55. Miyamoto, S., Seufzer, B. J., and Shumway, S. D. (1998) Mol. Cell. Biol. 18, 19-29

56. Lam, L. T., and Bresnick, E. H. (1996) J. Biol. Chem. 271, 32421-32429

57. Bresnick, E. H., and Felsenfeld, G. (1993) J. Biol Chem 268, 18824-18834.

58. Yasumoto, K., Okamoto, S., Mukaida, N., Murakami, S., Mai, M., and Matsushima, K. (1992) J. Biol. Chem. 267, 22506-22511

59. Kunsch, C., and Rosen, C. A. (1993) Mol. Cell. Biol. 13, 6137-6146

60. Mukaida, N., Okamoto, S., Ishikawa, Y., and Matsushima, K. (1994) J. Leukoc. Biol. 56, 554-558

61. Roebuck, K. A. (1999) J. Interferon Cytokine Res 19, 429-438

62. Yang-Yen, H. F., Chambard, J. C., Sun, Y. L., Smeal, T., Schmidt, T. J., Drouin, J., and Karin, M. (1990) Cell 62, 1205-1215

63. Forsberg, E. C., Johnson, K., Zaboikina, T. N., Mosser, E. A., and Bresnick, E. H. (1999) J. Biol Chem 274, 26850-26859.

64. Angel, P., Imagawa, M., Chiu, R., Stein, B., Imbra, R. J., Rahmsdorf, H. J., Jonat, C., Herrlich, P., and Karin, M. (1987) Cell 49, 729-739

65. Hu, E., Mueller, E., Oliviero, S., Papaionnou, V. E., Johnson, R., and Spiegelman, B. M. (1994) EMBO J. 13, 3094-3103

66. Gutman, A., and Wasylyk, B. (1990) EMBO J. 9, 2241-2246

67. Bidder, M., Loewy, A. P., Latifi, T., Newberry, E. P., Ferguson, G., Willis, D. M., and Towler, D. A. (2000) Biochemistry 39, 8917-8928

68. Ito, C. Y., Kazantsev, A. G., and Baldwin, A. S. (1994) Nucleic Acids Res. 22, 3787-3792

69. Kato, H., Taniguchi, Y., Kurooka, H., Minoguchi, S., Sakai, T., Nomura-Okazaki, S., Tamura, K., and Honjo, T. (1997) Development 124, 133-141

70. Roehl, H., Bosenberg, M., Blelloch, R., and Kimble, J. (1996) EMBO J. 15, 7002-7012

71. Nakielny, S., and Dreyfuss, G. (1997) Curr. Opin. Cell Biol. 9, 420-429

72. Lewis, T. S., Shapiro, P. S., and Ahn, N. G. (1998) Adv. Cancer Res. 74, 49-139

73. Deftos, M. L., and Bevan, M. J. (2000) Curr. Opin. Immunol. 12, 166-172

74. Osborne, B. A. (2000) Curr. Opin. Immunol. 12, 301-306

75. Robey, E. (1999) Ann. Rev. Immunol. 17,283-295

76. Kamei, Y., Xu, L., Heinzel, T., Torchia, J., Kurokawa, R., Gloss, B., Lin, S. C. , Heyman, R. A., Rose, D. W., Glass, C. K., and Rosenfeld, M. G. (1996) Cell 85, 403-414

77. Fronsdal, K., Engedal, N., Slagsvold, T., and Saatcioglu, F. (1998) J. Biol. Chem. 273, 1853-1859

78. Diamond, M. I., Miner, J. N., Yoshinaga, S. K., and Yamamoto, K. R. (1990) Science 249, 1266-1272

79. Rogatsky, I., Zarember, K. A., and Yamamoto, K. R. (2001) EMBO J. 20, 6071-6083

80. Benbow, U., and Brinkerhoff, C. E. (1997) Matrix Biology 15, 519-526

81. Kwok, S. F., Solano, R., Tsuge, T., Chamovitz, D. A., Ecker, J., Matsui, M., and Deng, T. (1998) Plant Cell 10, 1779-1790

What is claimed is:

1. A modulating agent for modulating AP-1 mediated cellular processes in a cell comprising an internalization moiety and one or more of:
(a) an isolated polypeptide from the intracellular domain of Notch-1 (NIC-1) which is capable of modulating an AP-1 mediated cellular process; or
(b) a peptide analog or peptidomimetic of the isolated polypeptide of (a) which is capable of modulating an AP-1 mediated response.

2. A modulating agent according to claim 1 wherein the isolated polypeptide comprises a RAM domain of NIC-1 (SEQ ID NO:1).

3. A modulating agent according to claim 1 wherein the isolated polypeptide comprises a partial RAM domain amino acid sequence set forth in SEQ ID NO:2.

4. A modulating agent according to claim 1 wherein the isolated polypeptide comprises a partial RAM domain amino acid sequence set forth in SEQ ID NO:3.

5. A modulating agent according to claim 1 wherein the isolated polypeptide comprises a partial RAM domain amino acid sequence set forth in SEQ ID NO:4.

6. A modulating agent according to claim 1 wherein the internalization moiety is a peptide localization signal.

7. A modulating agent according to claim 1 wherein the internalization moiety is a liposome.

8. A pharmaceutical composition comprising a pharmaceutically acceptable carrier and a modulating agent according to claim 1.

9. A method for modulating AP-1 mediated cellular processes in a cell, comprising contacting a cell with a composition including one or more of:
(a) an isolated polypeptide from the intracellular domain of Notch-1 (NIC-1) which is capable of modulating an AP-1 mediated cellular process; or
(b) a peptide analog or peptidomimetic of the isolated polypeptide of (a) which is capable of modulating an AP-1 mediated response; and thereby modulating an AP-1 mediated cellular process in the cell.

10. A method according to claim 9 wherein the isolated polypeptide comprises a RAM domain of NIC-1 (SEQ ID NO:1).

11. A method according to claim 9 wherein the isolated polypeptide comprises a partial RAM domain amino acid sequence set forth in SEQ ID NO:2.

12. A method according to claim 9 wherein the isolated polypeptide comprises a partial RAM domain amino acid sequence set forth in SEQ ID NO:3.

13. A method according to claim 9 wherein the isolated polypeptide comprises a partial RAM domain amino acid sequence set forth in SEQ ID NO:4.

14. A method according to claim 9 wherein the composition further includes an internalization moiety.

15. A method according to claim 14 wherein the internalization moiety is a peptide localization signal.

16. A method according to claim 14 wherein the internalization moiety is a liposome.

17. A method according to claim 9 wherein the modulating agent is present within a pharmaceutical composition comprising a pharmaceutically acceptable carrier.

18. A method according to claim 9 wherein the modulating agent is encoded by a recombinant nucleic acid present within the cell.

19. A method of treating a disease state in a patient including the step of administering to the patient a therapeutically-effective amount of a modulating agent according to claim 1.

20. A method of preventing or alleviating an inflammatory response in a patient including the step of administering to the patient a therapeutically-effective amount of a modulating agent according to claim 1.

21. A method of identifying a Notch-1 intracellular (NIC-1) domain-derived modulating agent effective in modulating AP-1 mediated transcription comprising the steps of:
(a) obtaining a cell line or organism transformed with a reporter gene operably linked to an AP-1 responsive element;
(b) contacting said transformed cell line or organism with:
(i) a polypeptide including a portion of the intracellular domain of Notch-1 (NIC-1); or
(ii) a peptide analog or peptidomimetic of the polypeptide of (i); and
(c) assaying the activity of the reporter gene wherein a statistically meaningful difference in the activity between the reporter gene in a transformed cell line or organism contacted with said portion of the NIC-1 domain, analog, or peptidomimetic thereof and the reporter gene in a transformed cell line or organism not contacted with said portion of the NIC-1 domain, analog, or peptidomimetic thereof correlates with the identification of a modulating agent.

22. A method according to claim 20 wherein the portion of the NIC-1 domain comprises at least one amino acid position mutated to differ from naturally-occurring NIC-1.


SEQUENCE LISTING (4 sequences)

SEQ ID NO: 1 - protein, 90 aa, Homo sapiens
RKRRRQHGQLWFPEGFKVSEASKKKRREPLGEDSVGLKPLKNASDGALMDDNQNEWGDEDLETKKFRFEEPVVLPDLDDQTDHRQWTQQH

SEQ ID NO: 2 - protein, 61 aa, Homo sapiens
RKRRRQHGQLWFPEGFKVSEASKKKRREPLGEDSVGLKPLKNASDGALMDDNQNEWGDEDL

SEQ ID NO: 3 - protein, 29 aa, Homo sapiens
LETKKFREEPVVLPDLDDQTDHRQWTQQH

SEQ ID NO: 4 - protein, 83 aa, Homo sapiens
RKRRRQHGQLWFPEGFKVSEASKKKRREPLGEDSVGLKPLKNASDGALMDDNQNEWGDEDLETKKFRFEEPVVLPDLDDQTDH